(19) United States
(12) Patent Application Publication
(10) Pub. No.: US 2017/0152306 A1
(43) Pub. Date: Jun. 1, 2017

(54) SINGLE CHAIN VARIABLE FRAGMENT (SCFV) ELONGATION MUTANTS

(71) Applicant: UNIVERSITAT AUTONOMA DE BARCELONA, (Cerdanyola del Vallès) (ES)

(72) Inventors: Sandra VILLEGAS, Bellaterra (Cerdanyola del Vallès) (ES); Geovanny RIVERA HERNANDEZ, Tuxtla Gutierrez (MX); Marta MARIN-ARGANY, Barcelona (ES); Bernat BLASO MORENO, Barcelona (ES)

(73) Assignee: UNIVERSITAT AUTONOMA DE BARCELONA, (Cerdanyola del Vallès) (ES)

(21) Appl. No.: 15/311,478

(22) PCT Filed: May 16, 2014
(86) PCT No.: PCT/EP2014/060047

§ 371 Date: Nov. 15, 2016

(51) Int. Cl. C07K 16/18

(52) U.S. Cl. CPC C07K 16/18; C07K 2317/622; C07K 2317/56; C07K 2317/24; A61K 2039/505

(57) ABSTRACT

The present invention relates to the provision of single chain variable fragment (scFv) elongation mutants. In addition, the present invention relates to a pharmaceutical composition comprising said scFv elongation mutants, as well as a nucleotide sequence encoding said scFv elongation mutants, a vector comprising said nucleotide sequence or a host cell expressing said nucleotide sequence. Particular embodiments relate to elongation mutants of the scFv-h3D6 which is derived from monoclonal antibody mAb-h3D6 (bapineuzumab). It is further disclosed a method of prevention or treatment of a patient in risk of suffering, or already diagnosted as suffering, Alzheimer disease that comprises the administration to said patient of an effective amount of the scFv elongation mutants of scFv-h3D6.

### FIELD OF THE INVENTION

The present invention relates to the provision of single chain variable fragments (scFv) elongation mutants. In addition, the present invention relates to a pharmaceutical composition comprising said scFv elongation mutants, as well as a nucleotide sequence encoding said scFv elongation mutants, a vector comprising said nucleotide sequence or a host cell expressing said nucleotide sequence.

### BACKGROUND TO THE INVENTION

In recent years, amyloid β (Aβ) immunotherapy has been considered a promising approach for Alzheimer disease (AD) treatment (cf. MAbs. 2009; 1:112-124; Immunotherapy 2012; 4:213-238; J. Neurochem. 2012; 120 Suppl. 1:186-193). In August 2012, however, Phase 3 trials of intravenously administered bapineuzumab were halted due to disappointing results (cf. The New York Times, 2012) with side effects of meningoencephalitis and cerebral amyloid angiopathy, the latter of which generated micro-hemorrage and vasogenic edema. In fact, in previous Phase 2 trials, adverse effects of bapineuzumab, a humanized monoclonal antibody (mAb-h3D6) were mostly mild and transient, but vasogenic edema occurred (cf. Lancet Neurol. 2010; 9:363-372). This side effect was dose-related and most cases were reported in apolipoprotein 4 (APOE4) carriers. Dose restriction and recruitment of only APOE4 non-carriers into another Phase 3 did not improve the outcome (cf. The New York Times, 2012; N. Engl. J. Med. 2014; 370(4):322-333). This failure does not imply that researchers should abandon the search for a molecule targeting and clearing Aβ oligomers. It is worth mentioning that these trials were performed too late in the disease course, as claimed in the World Alzheimer Report 2011 (cf. http://www.alz.co.uk/research/WorldAlzheimerReport2011.pdf). It is also plausible that drawbacks to the use of full-length antibodies could be prevented by using an antibody fragment that does not contain the Fc region, which is responsible for the activation of the microglia (cf. Nature 1999; 400:173-7), whereby activation of the microglia activates meningoencephalitis (inflammation). In contrast to the use of complete antibodies, administration of single-chain variable fragments (scFv) has not been associated with either meningoencephalitis or cerebral hemorrhage. Therefore, the use of single-chain variable fragments (scFv) has been proposed as a hopeful therapeutic strategy (cf. J. Neuroimmunol. 2000; 106:23-31; Biochem. Biophys. Res. Commun. 2006; 344:79-86; J. Mol. Biol. 2008; 384:917-928; Protein Eng. Des. Sel. 2009; 22:199-208; Expert Opin. Biol. Ther. 2011; 11:343-357; Biochem. J. 2011; 437:25-34).

Single chain variable fragment scFv-h3D6 (SEQ.ID.NO.: 1) is derived from the whole monoclonal antibody mAb-h3D6.v2 (cf. WO 2006066171 A1; Lancet Neurol. 2008; 7:805-811; Curr. Alzheimer Res. 2011; 8:808-817). The recombinant expression and aggregation pathway of scFv-h3D6 (SEQ.ID.NO.: 1) has been previously described (cf. Biochem. J. 2011; 437:25-34). Said single chain variable fragment scFv-h3D6 (SEQ.ID.NO.: 1) inhibits amyloid fibril formation and cytotoxicity of the Aβ₁₋₄₂-peptide. Addition of scFv-h3D6 completely precluded the toxic effect of Aβ-oligomers in the SH-SY5Y neuroblastoma cell line (cf. FIG. 15, and Marta Marin-Argany, Doctoral Thesis, Universitat AutOnoma de Barcelona, 2013; Biochem. J. 2011; 437:25-34). The study of the conformational properties of scFv-h3D6 upon heat treatment showed a conformational reorganization of the native state at ˜60° C. that led to the formation of an aggregation-prone intermediate state. This aggregation pathway did not correspond to an amyloid fibril pathway, as that followed by the Aβ-peptide, but rather to a worm-like fibril pathway which, noticeably, turned out to be non-toxic. In addition, this pathway was thermodynamically and kinetically favored when the scFv-h3D6 and Aβ₁₋₄₂-oligomers formed a complex in native conditions, explaining how the scFv-h3D6 withdraws Aβ₁₋₄₂ oligomers from the amyloid pathway in cell culture.

The high-yield recombinant production of scFvs is limited by their folding and stability properties. Thus, it is the purpose of the present invention to provide an isolated single chain variable fragment that precludes the toxic effect of Aβ-peptides (preferably by driving Aβ-oligomers to the non-toxic worm-like pathway), yet also exhibits increased thermodynamic stability, lower aggregation tendency and an increased half-life of said scFv. It is additionally the problem of the present invention to provide an isolated scFv which is able to be produced in pharmaceutical-scale quantities and which exhibits improved safety in the prevention and/or treatment of Alzheimer's disease due to it being able to be administered in reduced doses. These technical features are achieved due to the elongation performed in scFv structure which improves scFv folding by increasing scFv thermodynamic stability and decreasing scFv aggregation, without losing scFv ability to bind Aβ peptides.

### BRIEF DESCRIPTION OF THE INVENTION

The present invention relates to an isolated single chain variable fragment comprising variable regions of the heavy (V_{H}) and light chains (V_{L}) of a monoclonal antibody, particularly of a human or humanized monoclonal antibody, characterized in that the C-terminal end of said light chain is elongated by:

i) an amino acid residue, or
ii) a polypeptide having at least 2 amino acid residues.

A further preferred embodiment of the present invention relates to an isolated single chain variable fragment comprising variable regions of the heavy (V_{H}) and light chains (V_{L}) of humanized monoclonal antibody mAb-h3D6, characterized in that the C-terminal end of said light chain is elongated by:
i) an amino acid residue, or
ii) a polypeptide having at least 2 amino acid residues,

Still more preferably, and the above mentioned scFv elongation mutant of mAb-h3D6 which functionally aggregates with the Aβ₁₋₄₂ peptide to form worm-like fibrils.

In one preferred embodiment the C-terminal end of the single chain variable fragment comprising variable regions of the heavy (V_{H}) and light chains (V_{L}) of humanized monoclonal antibody mAb-h3D6 is an amino acid residue present in beta-strand G. More preferably, that C-terminal end amino acid corresponds to lysine residue 107 of the light chain (V_{L}-K107) of the wild-type mAb-h3D6 using the Kabat Numbering Scheme.

In another preferred embodiment, the C-terminal end of said single chain variable fragment of the invention is elongated by a glycine residue, an arginine or a lysine residue. As preferred embodiment for present invention the amino acids used for elongating scFv, either residue by residue (a) or as members of a polypeptide (b) are non-antigenic for mammals, preferably, for human beings.

Alternatively, the C-terminal end of said single chain variable fragment of the invention is elongated by an arginine-threonine (Arg-Thr) dipeptide. For the purposes of the present description the term “dipeptide” means a peptide formed from 2 amino acid residues. Other alternative elongation dipeptides are selected from the following pairs of amino acids: 108Gly 109Gly; 108Gly 109Thr; 108Gly 109Ser; 108Arg 109Gly; 108Arg109Thr; 108Arg 109Ser; 108Lys 109Gly; 108Lys109Thr; 108Lys 109Ser. Before each amino acid the position in the light chain (V_{L}) is indicated. Although, any amino acid, particularly conservative substitutions of the amino acids indicated, can form part of the elongation polypeptides of present invention.

According to present invention, the formation of worm-like fibrils is identified by the appearance of a spectral peak at between 1618 and 1628 nm in the Fourier Transform Infrared (FTIR) spectrum.

The single chain variable fragment of the invention is represented by SEQ.ID.NO: 2, SEQ. ID. NO: 3 or SEQ.ID.NO: 4. For the purposes of the present description single chain variable fragments elongation mutants of the invention can be also represented by amino acid sequences having identity >70% and/or homology >90% to SEQ.ID.NO: 2, SEQ. ID. NO: 3 or SEQ.ID.NO: 4, providing the amino acids elongating the C-terminal end were conserved or replaced by conservative substitutions, including substitution of arginine, threonine or lysine, by glycine. Particularly preferred amino acid substitutions a Lys by Arg (and viceversa); and Thr by Ser (and viceversa).

Present invention also covers nucleotide sequences encoding the single chain variable fragments elongation mutants disclosed above; the vectors comprising the same and the host cells expressing said nucleotide sequences.

Moreover, the invention also relates to isolated single chain variable fragments elongation mutants as disclosed above for use as medicament. More precisely, isolated single chain variable fragments elongation mutants as disclosed hereto, for use in the prevention and/or treatment of Alzheimer's disease in a patient.

Furthermore, the invention also comprises the use of the isolated single chain variable fragments elongation mutants disclosed previously, in the manufacture of a medicament for the prevention and/or treatment of Alzheimer's disease.

Finally, the invention also covers pharmaceutical compositions comprising at least the isolated single chain variable fragments elongation mutants disclosed previously as active ingredient and, optionally, at least a second active ingredient and/or al least an inert ingredient such an excipient and/or carrier.

### BRIEF DESCRIPTION OF THE FIGURES

FIG. 1. Secondary structure of the wild-type scFV-3D6h (WT, SEQ.ID.NO.: 1). Far-UV CD-spectra in different urea concentrations. (A) 0-6 M urea. 0 M, black; 2.2 M urea, dark grey; 3.7 M urea, mild gray; 4.8 M, light grey; 6.0 M, faint gray. The spectrum of the native state shows two minima (218 nm and 230 nm), a maximum (200 nm, not shown), and a positive shoulder (237 nm). The initial minimum at 230 nm is maintained until 3.7 M urea. (B) 6-8.6 M urea. 0 M, black; 6.0 M urea, dark grey; 6.9 M urea, mild gray; 8.0 M, light grey; 8.6 M, faint gray. The positive shoulder at 237 nm is lost between 6.9 and 8 M urea, and a pure random-coil conformation is not achieved even at 8.6M urea.

FIG. 2. Urea denaturation of scFV-3D6h wild type [WT (SEQ.ID.NO.: 1)] and its elongation mutants [C1 (SEQ.ID.NO.: 2), C2 (SEQ.ID.NO.: 3), C3 (SEQ.ID.NO.: 4)]. The maximum of the tryptophan-fluorescence emission-spectra is plotted as a function of urea concentration. (A-D) Fitting to the three-state model. (A) WT, (B) C1, (C) C2, (D) C3. E) Comparison of the fitted data. Two transitions are observed during the red-shift. The plateau in the 4-6M urea region is indicative of the occurrence of an intermediate state. (F) Magnification of the first transition shows incremented thermodynamic stability upon mutation. WT, black; C1, dark grey; C2, mild gray; C3, light grey.

FIG. 3. Limited proteolysis with thermolysin of the urea-induced intermediate analyzed by SDS-PAGE. (A) Proteolysis of the WT at 5 M Urea (elongation mutants gave the same result, not shown). N-terminal sequencing of the blotted main-band (squared) rendered the sequence of the V_{H} domain (GAMEVQ). (B) Kinetics of proteolysis at 3 M urea is different among elongation mutants. WT, black; C1, dark grey; C2, mild gray; C3, light grey.

FIG. 4. ScFv-h3D6 3D-model. (A) 3D-model. CDRs, blue; linker, black; tryptophan (Trp) residues, red; disulphide bridges, yellow. (B)C-terminal end detail showing the main interactions between V_{L}-E105 and V_{L}-K107, and the effect of OXT107-O.

FIG. 5. Far-UV CD spectra of the scFV-3D6h elongation mutants in native conditions. WT, black; C1, dark grey; C2, mild gray; C3, light grey.

FIG. 6. Thermal denaturation of scFV-3D6h WT and its elongation mutants. (A) Ellipticity at 218 nm. (B) tryptophan-Fluorescence emission at 338 nm. WT, black; C1, dark grey; C2, mild gray; C3, light grey.

FIG. 7. Deconvolution and comparison of some FTIR spectra. (A) Native WT (25° C.). (B) Renaturalized WT. (C) Renaturalized C1 (D) Renaturalized C2. (E) Renaturalized C3. (A-E) Band corresponding to native β-sheet is dotted and bands corresponding to fibril β-strand components are in black. (F) Comparison of spectra. Native WT, dotted; renaturalized WT black; renaturalized C1, dark grey; renaturalized C2, mild gray; renaturalized C3, light grey.

FIG. 8. Molecular structure of β-amyloid fibrils in Alzheimer's disease brain tissue. The three dimensional structure of these oligomers extracted from human is based on trimeric stacks leaving the N-terminal residues 1-5 solvent exposed (refer also to Cell 2013; 154(6):1257-1268).

FIG. 9. Scheme of the wild-type scFv-h3D6 sequence and Kabat numbering. The V_{H} domain is 119 residues long and contains six insertions (a-e), and therefore ends at residue 113 using Kabat numbering. The V_{L} domain is 112 residues long and contains five insertions, and therefore ends at residue 107 using Kabat numbering. The V_{H} domain contains three tryptophan residues, whereas the V_{L} domain contains two (W, red). Both domains are folded in 9 canonical β-chains (VH-βA, B, C, C′, C″, D, E, F and G) and contain a disulphide bridge (yellow). The six CDR regions (VH, green; VL, blue) mainly correspond to regions of irregular loops. The flexible linker (Gly₄Ser)₃ linking both domains is shown in black.

FIG. 10. Immunoblotting analysis of the soluble Aβ-amyloid oligomers from extracellular extracts of 5 month-old NTg and 3×Tg-AD mice. (A) Extracellular extracts from several brain subregions (HC, hippocampus; CX, cortex; OB, olfactory bulb and CR, cerebellum), from non-transgenic (NTg) and triple transgenic (3×Tg-AD) mice I.P. treated with 85 μg of scFv-h3D6 (+) and I.P. treated with PBS (−), were analyzed. Profiles for NTg mice did not change upon scFv-h3D6 treatment. The profiles for 3×Tg-AD mice of extracellular soluble Aβ oligomers in CX and OB showed a clear decrease of the dodecameric, nonameric, hexameric and trimeric Aβ-species upon treatment (squared), while in HC and CR remained the same. Arrows indicate respective migration position of monomers (1-mer), trimers (3-mer), hexamers (6-mer), nonamers (9-mer), dodecamers (12-mer) and sAPP (secreted form of APP that has been cleaved by either α- or β-secretase). Synthetic human Aβ1-42 peptide (hAβ42) was used as a positive control (left lanes). Total protein applied to each lane was 45 μg. Blots were normalized by β-actin concentration. (B) Bar diagram showing the mean±SD of the quantification of the bands from three experiments.

FIG. 11. Clusterin (A) and apoE (B) concentrations in TBS extracts determined by ELISA. Mean±SD, n=5, Mann-Whitney U test, *p<0.05 vs. untreated NTg mice. #p<0.05 vs. untreated 3×Tg-AD mice.

FIG. 12. (A) Depletion of deep cerebellar nuclei neurons in the 3×Tg-AD mouse cerebellum and recovery by scFv-h3D6 treatment. Cell numbers from fastigial, interposed and dentate nuclei were determined. Black, untreated NTg group; Striped, scFv-treated NTg group; White, untreated 3×Tg-AD group; Squared, scFv-treated 3×Tg-AD group. Results are expressed by means±SEM *significant vs. untreated NTg group (p s 0.05); **significant vs. untreated 3×Tg-AD group (p<0.03). Significance values were calculated via Mann-Whitney test. (B) Illustrative photomicrographs of sagittal sections. At the level of the fastigial and interpositus nuclei the involvement of DCN neurons and its protection by scFv-h3D6 is shown. Bar is 50 μm.

FIG. 13. (A) Aβ detection by 6E10 mAb in cerebral cortex (large pyramidal neurons). In red, βA detected by 6E10 mAb. In blue, DAPI staining of the nuclei. a, untreated NTg mice; b, treated NTg mice; c, untreated 3×Tg-AD mice; d, treated 3×Tg-AD mice. (B) Aβ detection by 6E10 mAb in the hippocampus (Pyramidal neurons from the CA2). In red, βA detected by 6E10 mAb. In blue, DAPI staining of the nuclei. a, untreated NTg mice; b, treated NTg mice; c, untreated 3×Tg-AD mice; d, treated 3×Tg-AD mice. (C) Aβ detection by 6E10 mAb in the Olfactory Bulb (mitral cells). In red, βA detected by 6E10 mAb. In blue, DAPI staining of the nuclei. a, untreated NTg mice; b, treated NTg mice; c, untreated 3×Tg-AD mice; d, treated 3×Tg-AD mice. Scale bar, 10 μm. (D) Aβ detection by 6E10 mAb in the cerebellum (macroneurons from the fastigial nucleus). In red, βA detected by 6E10 mAb. In blue, DAPI staining of the nuclei. a, untreated NTg mice; b, treated NTg mice; c, untreated 3×Tg-AD mice; d, treated 3×Tg-AD mice.

FIG. 14. Gene map of pETtrx_1a (SEQ.ID:NO.: 11).

FIG. 15. Amyloid fibril formation by Aβ42 fibers WL, scFv and scFv-Aβ42 complex. (A) 100 μM Aβ42 (B) 100 μM scFv-h3D6 (C) 100 μM scFv-h3D6 in the absence of DMSO (D) 100 μM Aβ42 with 100 μM scFv-h3D6, incubated 48 hours at 37° C. and 5 min at 90° C. (A) The cytotoxic peptide Aβ oligomers are observed at 37° C. and amyloid fibrils obtained at 90° C. (B) The scFv-WL h3D6 does not form fibers in the presence of DMSO. (C) The scFv-h3D6 in the absence of DMSO does not form WL fibrils at 37° C., but it dues at temperatures higher than 60° C. (were the intermediary state is most populated), as at 90° C. (D) The Aβ42:scFv-h3D6 complex forms WL fibrils at 37° C. just upon mixing both proteins. Heating to 90° C. leads to dissociation of the complex.

### DETAILED DESCRIPTION OF THE INVENTION

The present invention relates to isolated single chain variable fragment (scFv) elongation mutants (Rivera-Hernandez et al. mAbs 5:5, 678-689 (2013). A scFv is a fusion protein of the variable regions of the heavy (V_{H}) and light chains (V_{L}) of at least one antibody. In the present invention, the scFv is a fusion protein comprising variable regions of the heavy and light chains of humanized monoclonal antibody mAb-h3D6. Preferably, the V_{H} domain is linked to the V_{L} domain by a (Gly₄Ser)₃ linker.

A humanized antibody is an antibody from a non-human species whose protein sequence has been modified to increase its similarity to antibody mutants produced naturally in humans. In the present invention, the humanized antibody is a humanized monoclonal antibody. A monoclonal antibody (mAb or moAb) is a monospecific antibody that is made by identical immune cells that are clones of a unique parent cell, whereby antibodies derived from said cells have monovalent affinity in that they bind to the same epitope as other antibodies derived from the same cells.

In the present invention, the humanized monoclonal antibody is preferably the 3D6 antibody mAb-h3D6, more preferably mAb-h3D6.v2, which exhibits affinity for the 1-5 N-terminal residues of amyloid-β polypeptide. The humanized monoclonal antibody mAb-h3D6.v2 exhibits a dissociation constant, K_{d}, for the amyloid-β polypeptide of 2.24 nM, as measured by BIAcore analysis. In comparison, non-humanised 3D6 has a K_{d} of 0.88 nM, and h3D6vl a K_{d} of 2.06 nM (cf. WO 2006066171 A1). An ELISA competitive binding assay using a biotinylated 3D6 antibody revealed an approximate 6-fold less binding affinity for h3D6vl and h3D6v2 in comparison to their non-humanized counterpart.

In the wild-type scFv, mAb-h3D6.v2, 64 amino acid residues out of 246 (26%) belong to the complementarity determining regions (CDRs) recognizing Aβ-oligomers. The remaining amino acid residues (74%) correspond to the framework regions (FRs). The FRs derive from those of IgG2b (the original in mAb-h3D6.v2), but other isotypes having different FRs may be employed. The wild-type scFV mAb-h3D6.v2 is encoded by SEQ.ID.NO.: 5.

The scFv of the present invention is preferably characterized in that

(a) the C-terminal end of said light chain is elongated by:
i) an amino acid residue, or
ii) a polypeptide comprising at least 2 amino acid residues, and
   (b) the single chain variable fragment aggregates with the Aβ₁₋₄₂ peptide to form worm-like fibrils.

The C-terminal end of the light chain is the end of the scFv polypeptide that is terminated by a free carboxyl group (—COOH) or substituted form thereof. In one embodiment of the invention, the C-terminal end is an amino acid residue present in beta-strand G. An amino acid residue is an amino acid that has been incorporated into a peptide and is characterised by loss of either a hydrogen atom from the amine end or a hydroxyl moiety from the carboxyl end, or both, to form such peptide. Beta-strand G is a strand of one of the two antiparallel β-sheets packed against each other in a compressed β-barrel in the immunoglobulin variable domains. In a preferred embodiment, the C-terminal end of the light chain is the end of the scFv polypeptide that corresponds to lysine residue 107 of the light chain (VL-K107 using the Kabat Numbering Scheme). Thus, the C-terminal end is marked by a —NHCH[(CH₂)₄NH₂]COOH moiety (or ionic, zwitterionic, or substituted forms thereof) which corresponds with lysine residue 107 of the scFv molecule.

In the present invention, the C-terminal end of the light chain is elongated with either an amino acid or a polypeptide comprising at least 2 amino acid residues. Thus, the free carboxylic acid moiety of the C-terminal end, preferably the free carboxylic acid moiety of the amino acid residue present in beta-strand G, more preferably the free carboxylic acid moiety of the amino acid residue corresponding to lysine residue 107 of the light chain (V_{L}-K107) of mAb-h3D6.v2, is elongated with an amino acid or a polypeptide comprising at least 2 amino acid residues via a peptide bond. As a consequence, the carboxylic acid of the amino acid residue at the C-terminal end of the light chain is bonded to the amine of an amino acid residue or the amine at the N-terminal end of a chain of at least 2 amino acid residues, more preferably between two and ten amino acid residues, yet more preferably two or three amino acid residues, most preferably 2 amino acid residues, via a peptide bond. The elongation of the C-terminal end of said light chain by, at least, one amino acid residue, or a polypeptide comprising at least 2 amino acid residues preferably results in an elongated scFv. The elongated scFv of the present invention is therefore an elongation mutant.

In one embodiment, the C-terminal end of the light chain is elongated with an amino acid residue, preferably selected from proteinogenic amino acid residues (defined as amino acid residues naturally occurring in proteins and preferably selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, selenocysteine, pyrrolysine and N-formylmethionine residues) and non-proteinogenic amino acids (defined as amino acid residues not naturally occurring in proteins and preferably selected from non-α amino acid residues such as β-alanine, GABA and 5-aminolevulinic acid residues; D-amino acid residues, such as D-alanine, D-glutamate, D-lysine and D-serine residues; amino acid residues lacking a H on the α-carbon such as α-amino isobutyric acid and dehydroalanine residues; and amino acid residues having twin stereocentres such as cystathionine, lanthionine, djenkolic acid and diaminopimelic acid residues). More preferably the amino acid residue is selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine. Furthermore preferably, the amino acid residue is an aliphatic amino acid (glycine, alanine, valine, leucine or isoleucine) residue, or a basic amino acid (Histidine, Lysine or Arginine) residue. In a preferred embodiment, the C-terminal end of the single chain variable fragment is substituted (elongated) by a glycine residue. In another preferred embodiment, the C-terminal end of the single chain variable fragment is substituted (elongated) by an arginine residue.

In a preferred embodiment of the invention, the C-terminal end of the light chain is lysine residue 107 which is elongated with a glycine residue (i.e. V_{L}-el-R108G herein referred to as C1, SEQ.ID.NO.: 2). In another embodiment, the C-terminal end of the light chain is lysine residue 107 which is elongated with an arginine residue (i.e. V_{L}-el-R108, herein referred to as C2, SEQ.ID.NO.: 3).

In another embodiment, the C-terminal end of the light chain is elongated with a polypeptide comprising at least 2 amino acid residues. Preferably the elongation polypeptide comprises a proteinogenic amino acid residue at the N-terminal end of said elongation polypeptide, more preferably an amino acid selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine. Yet more preferably, the amino acid residue at the N-terminal end of said polypeptide is a basic amino acid residue (selected from Histidine, Lysine or Arginine), furthermore preferably an arginine residue.

In another preferred embodiment, the C-terminal end of the light chain is lysine residue 107 which is elongated with a dipeptide comprising an arginine residue at its N-terminal end and a threonine residue at its C-terminal end (i.e. V_{L}-el-R108T109, herein referred to as C3, SEQ.ID.NO.: 4).

The formation of the elongating amino acid or peptide can be carried out by chemical synthesis, by a biotechnology process, including mutagenesis. Preferably elongation is achieved by extending the DNA sequence of the wild-type scFV before the Stop codon so that it encodes the elongation mutant which may then be transcribed. Alternatively, elongation can be achieved in a single step process, amino acid by amino acid, or, in the case of adding an elongating peptide, particularly a dipeptide, to the C-terminal end of the single chain variable fragment, also in one-pot step, adding both amino acids linked together.

The N-terminal end of the light chain (V_{L} domain) is linked to the C-terminal end of the heavy chain (V_{H} domain), preferably by a (Gly₄Ser)₃ linker. In addition, the N-terminal end of the V_{H} domain is preferably terminated by a tripeptide sequence GAM (glycine-alanine-methionine), whereby the methionine residue at the C-terminal end of said tripeptide is linked to the N-terminal end of the V_{H} domain via a peptide bond. The tripeptide sequence GAM, used with expression vector pET-trx-1a, may be replaced with an alternative sequence, depending on the expression vector employed. Neither GAM tripeptides, nor the corresponding DNA bases encoding the same, are shown in the corresponding sequences enclosed hereto in the sequence listing attached to present specification, for the elongation mutants of present invention.

The scFv of the present invention preferably comprises a protein tag. A protein tag is a peptide sequence genetically grafted onto a recombinant protein for a purpose. In the present invention, said protein tag is a solubilization tag to assist in the proper folding of said protein and keep it from precipitating while in solution. Said solubilization tag is especially useful in improving the aqueous solubility of recombinant proteins expressed in chaperone-deficient species such as E. coli. Preferably the solubilization tag is selected from NusA, thioredoxin (TRX), poly(NANP), maltose binding protein (MBP), or glutathione-S-transferase, more preferably TRX. Alternatively, the sequence of the scFv of the present invention (SEQ ID NO: 1) may be cloned in a periplasmic expression vector without any tag, but rather with a signal peptide. Similarly, the elongation mutants of the present invention may be cloned in Pichia pastoris, or another eukaryotic expression system such as COS or baculovirus,

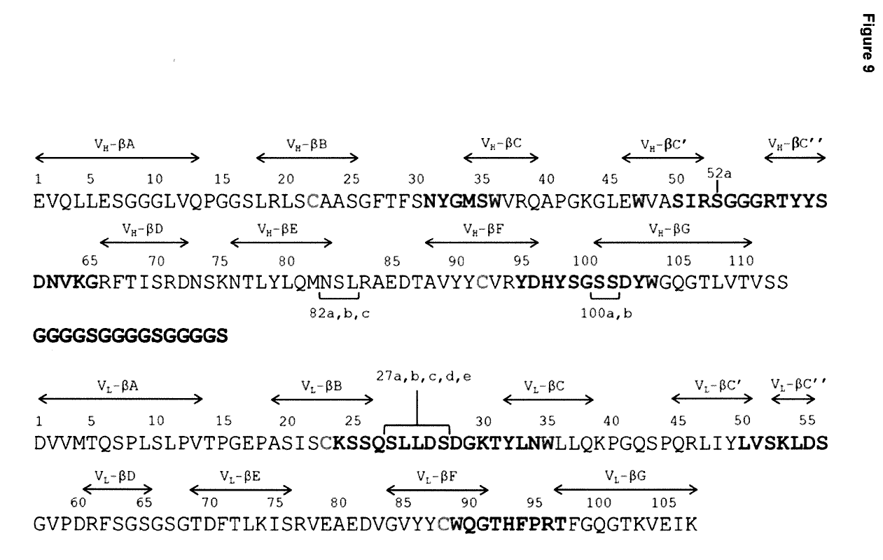

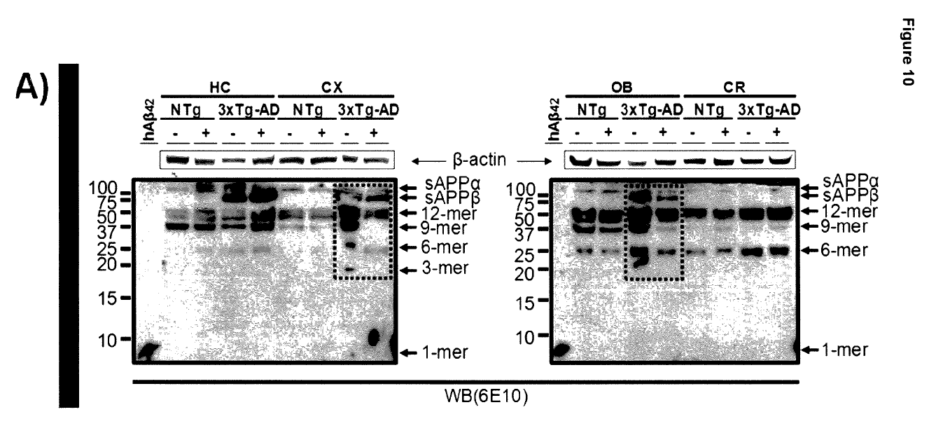

The scFv of the invention aggregates with Aβ peptides. Aβ peptides are the main component of neuritic amyloid plaques which comprise a tangle of regularly ordered fibrillar aggregates called amyloid fibers. Preferably the Aβ peptides are fibrillogenic (fibril-generating and hence amyloid plaque-generating) peptides. Preferably, the Aβ peptides can aggregate to form Aβ oligomers which take the form of multiples of trimeric Aβ peptides, more preferably trimers, hexamers, nonamers and dodecamers (cf. FIG. 10). In particular, the ScFv of the present invention is further characterized in that it aggregates with the Aβ₁₋₄₂ peptide. This aggregation of the scFv and the Aβ₁₋₄₂ peptide is a protein aggregation in which misfolded proteins aggregate (i.e. accumulate and clump together) either intra- or extracellularly. The scFv of the invention complexes with Aβ peptides, not only preventing the formation of Aβoligomers and toxicity thereof (i.e. the scFv of the invention inhibits Aβ peptide amyloid fibril formation and cytotoxicity), but also removing those Aβ oligomers that have already been formed in vivo (cf. FIG. 10). In the present invention aggregation of the scFv and the Aβ₁₋₄₂ peptide is characterized by the formation of worm-like fibrils. Worm-like fibrils are fibrils (aggregates) which have a short and curved (worm-like) appearance when visualized by transmission electron microscopy (TEM) and which are different from the amyloid fibrils formed by the Aβ peptides. The formation of worm-like fibrils may also be identified by the appearance of a spectral peak or component at between 1618 and 1628 nm, more preferably between 1619 and 1627 nm, furthermore preferably between 1620 and 1627 nm, furthermore preferably centered at 1626 nm in the Fourier Transform Infrared (FTIR) spectrum.

In order that the elongation mutants of the present invention aggregates with Aβ peptides, said elongation mutants preferably do not comprise non-native folding, such as that caused by incorrect disulfide bridge formation. In addition, said elongation mutants preferably do not induce an antigenic or allergic reaction in humans or animals to which it is administered. Preferably, the C-terminal end of the elongated scFv therefore ends in a glycine, arginine, lysine, threonine or serine residue, more preferably a glycine, threonine, arginine or lysine residue, yet more preferably a glycine, threonine or arginine residue.

The scFv of the invention (i.e. the elongation mutants of the invention) is an isolated scFv, meaning that it has been isolated from organic matter, preferably from organic polymers, yet more preferably from endotoxins and exotoxins (i.e. pyrogens), furthermore preferably from lipopolysaccharides, to a level of 99% purity. Preferably the isolated scFv is a scFv which has been purified by removing lipopolysaccharides from the protein to a level of purity of 99.9%, yet more preferably 99.99%, furthermore preferably 99.999%. Lipopolysaccharides are the major endotoxins of Gram-negative bacteria, and they could elicit a cellular response in assays with cells or animals, or in prevention and/or treatment of a disease in a patient. Preferably lipopolysaccharides were removed from the protein by using ion-exchange chromatography, distillation and/or ultrafiltration, preferably using ion-exchange chromatography.

The three elongation mutants—C1, C2, C3—used as examples of present invention, they all three improved folding, resulting in increased thermodynamic stability and lower aggregation tendency, as determined by urea-denaturation experiments and Fourier-transform infrared spectroscopy (FTIR), respectively, without loss of the ability to bind to the Aβ-oligomers, when compared with the wild-type scFv. Moreover, because the elongation mutants maintained the ability to drive Aβ-oligomers to the non-toxic worm-like pathway, these traits increase the half-life of scFv-h3D6 and, consequently, decrease the effective doses in the same mouse model in which the original form has proven to be beneficial (cf. Villegas S., Rivera-Hernandez G., Cervera L., Marin-Argany M., Blasco-Moreno B., & Giménez-Llort L. Effects of scFv-h3D6 on 3×Tg-AD mouse model of Alzheimer's disease: Cognitive improvement and modification of the Aβ-peptide aggregation pathway.; MAbs. 2013; 5(5): 660-664; MAbs. 2013; 5(5): 665-677). As a result, the isolated single chain variable fragment elongation mutants of the present invention not only precludes the toxic effect of Aβ-peptides, but also exhibits increased thermodynamic stability, lower aggregation tendency and an increased half-life of said scFv. Thus, said scFv is able to be produced in pharmaceutical-scale quantities and is administered in decreased doses for the prevention and/or treatment of Alzheimer's disease, thereby improving its safety and reducing costs to the patient.

The scFv elongation mutants of the present invention may be obtained on an industrial scale by expression of said protein in a transgenic organism such as bacteria or yeast (fungi). Preferably protein expression is in bacteria such as E. coli, non-pathogenic species of the gram-positive Corynebacterium (e.g. C. glutamicum), or Pseudomonas fluorescens, more preferably in E. coli using expression of a Trx-fusion precursor. Alternatively, the scFv of the present invention may be obtained from yeast such as Saccharomyces cerevisiae or Pichia Pastoris, or filamentous fungi such as Aspergillus spp., Trichoderma spp. or Myceliophthora thermophila, or other expression system such as COS or baculovirus, more preferably from Pichia pastoris. Preferably the scFv of the present invention is obtained on an industrial scale in a bioreactor of between 100 mL and 100 L volume, preferably between 0.5 and 10 L in volume.

The present invention also comprises a nucleotide sequence encoding the single chain variable fragment elongation mutants according to the present description. Preferably the nucleotide sequence is any of the nucleotide sequences SEQ.ID.NO.: 6, SEQ.ID.NO.: 7 or SEQ.ID.NO.: 8.

The present invention also comprises a vector comprising the nucleotide sequence according to the present description. Preferably the vector is encoded by the nucleotide sequence SEQ.ID.NO.: 11. Further disclosed is a host cell expressing said nucleotide sequence.

The present invention also comprises an elongated single chain variable fragment elongation mutants according to the present description for use as medicament in neurodegenerative diseases, light chain amyloidosis and other systemic amyloidosis, cancer and auto-immune diseases preferably in the prevention and/or treatment of Alzheimer's disease in a patient.

Moreover, the present invention also comprises a pharmaceutical composition comprising at least an isolated single chain variable fragment elongation mutants according to that disclosed herein and, optionally, at least a second active ingredient and/or at least an inert ingredient such an excipient and/or carrier. Said excipient or carrier may be a diluent, as a way of example. Such compositions can be in the form of a capsule, sachet, paper or other container. In making the compositions, conventional techniques for the preparation of pharmaceutical compositions may be used. For example, the compound of interest will usually be mixed with a carrier, or diluted by a carrier, or enclosed within a carrier that may be in the form of an ampoule, capsule, sachet, paper, or other container. When the carrier serves as a diluent, it may be solid, semi-solid, or liquid material that acts as a vehicle, excipient, or medium for the active compound. The compound of interest can be adsorbed on a granular solid container for example in a sachet. Some examples of suitable carriers are water, salt solutions, alcohols, polyethylene glycols, polyhydroxyethoxylated castor oil, peanut oil, olive oil, lactose, terra alba, sucrose, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose, and polyvinylpyrrolidone. Similarly, the carrier or diluent may include any sustained release material known in the art, such as glyceryl monostearate or glyceryl distearate, alone or mixed with a wax. The formulations may also include wetting agents, emulsifying and suspending agents, preserving agents, sweetening agents or flavouring agents. The formulations of the invention may be formulated so as to provide quick, sustained, or delayed release of the active ingredient after administration to the patient by employing procedures well known in the art. In addition, the pharmaceutical compositions can be sterilized and mixed, if desired, with auxiliary agents, emulsifiers, salt for influencing osmotic pressure, buffers and/or colouring substances and the like, which do not deleteriously react with the scFv of the invention.

Finally, the invention also relates to a new method of prevention or treatment of a patient in risk of suffering, or already diagnosted as suffering, Alzheimer disease, that comprises the administration to said patient of an effective amount of the scFv elongation mutants of present invention or of an pharmaceutical composition comprising the same.

Effective amount of the elongation mutants of the invention or of a composition comprising the same, particularly of a pharmaceutical or nutraceutical composition, for the purposes of present specification, is meant any dose, administered throughout any route (oral, transdermal or subcutaneous injection, dialysis, etc. . . . ), to a patient which prevented or ameliorated Alzheimer disease symptoms (at behavioural or physiological level), particularly shown as increasing clearing Aβ oligomers, and/or inhibition of amyloid fibril formation and/or cytotoxicity of Aβ₁₋₄₂-peptide. Moreover, the scFv elongation mutants of present invention can be administered for prevention or treatment of Alzheimer disease either, as sole active principles, optionally in formulation with at least an inert ingredient, such an excipient or carrier; or in combination, simultaneously or under separate combined administration, with at least another active principle.

Various modifications of the invention in addition to those shown and described herein will become apparent to those skilled in the art from the foregoing description and fall within the scope of the appended claims. It is understood that the application of the teachings of the present invention to a specific problem or situation will be within the capabilities of one having ordinary skill in the art in light of the teachings contained herein. Examples of the products of the present invention and representative processes for their isolation, use, and manufacture appear below, but should not be construed to limit the invention.

### EXAMPLES

A) Materials and methods

### 1. Protein Expression and Mutagenesis.

Protein expression in the form of a Trx-fusion precursor was performed as reported in Biochem. J. 2011; 437:25-34.

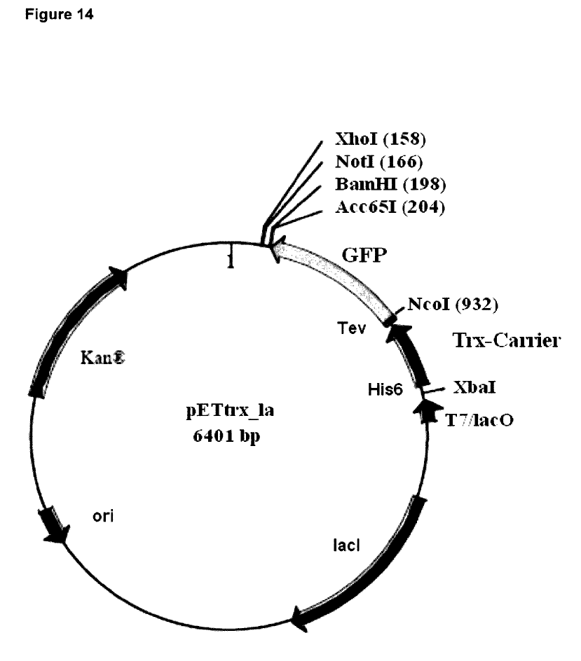

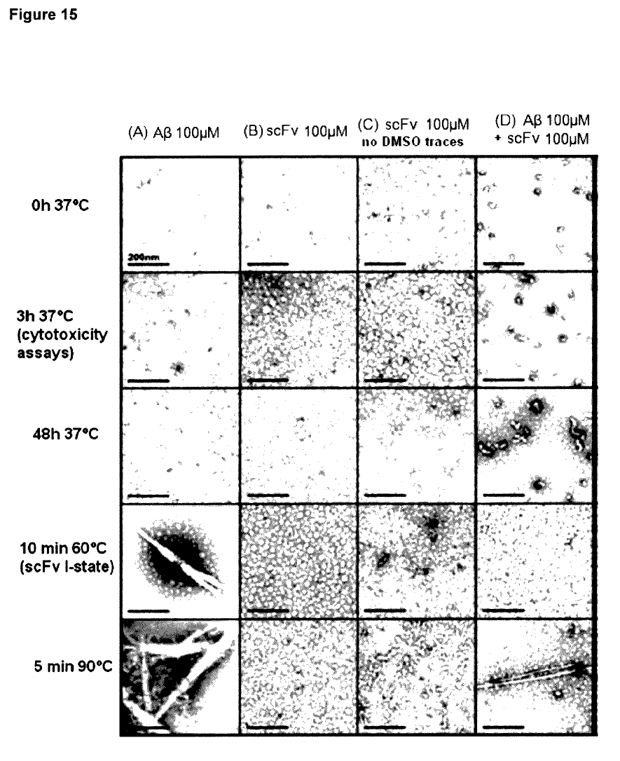

For large-scale production, the intracellular expression in pETtrx-1a (cf. FIG. 14) allowed for the purification of both soluble and insoluble fractions. Induction with 0.5 mM IPTG (isopropyl β-D-thiogalactopyranoside) was performed at D=0.7 and incubation in the shaker at 20° C. for 15 h. The cellular pellet was then washed three times with cold PBS (pH 7.4) and resuspended in Ni²⁺-binding buffer (20 mM sodium phosphate, 0.5 M NaCl and 0.5 mM EDTA, pH 7.4) containing a cocktail of protease inhibitors [1 μg/ml leupeptin, 1 μg/ml benzamidine, 1 μg/ml BPTI (basic pancreatic trypsin inhibitor) and 1 mM PMSF]. After two freeze-thaw cycles, the sample was sonicated for 5 cycles of 45 s, at 50% duty cycle and output 9 (Sonifier 450, Branson). The soluble and the insoluble fractions were fractionated at 43700 g. The soluble fraction, containing a 40.6 kDa precursor (His6-thioredoxin-TEV target-scFv), was purified by 5 ml Histrap HP columns (GE Healthcare). The presence of 0.5 mM EDTA was necessary to preclude undesirable proteolysis within the time-course of the IMAC (immobilized metal-ion-affinity chromatography). This precursor was also obtained by solubilizing the insoluble fraction in denaturing buffer (100 mM Tris/HCl, 10 mM GSH, pH 8.5, and 8 M urea) and refolding by dilution (1:10) in ice-cold refolding buffer (100 mM Tris/HCl, 100 mM L-arginine and 0.15 mM GSSG, pH 8.5) for 48 h. The precursor was proteolized at 30° C. with TEV protease for 4 h at a precursor/protease ratio of 50:1 (w/w) in 20 mM Tris/HCl, 100 mM NaCl, 0.5 mM EDTA, 0.3 mM GSSG and 3 mM GSH, pH 8.3 [Nallamsetty, S., Kapust, R. B., Tozser, J., Cherry, S., Tropea, J. E., Copeland, T. D. and Waugh, D. S. (2004) Efficient site-specific processing of fusion proteins by tobacco vein mottling virus protease in vivo and in vitro. Protein Expr. Purif. 38, 108-115]. TEV protease was recombinantly obtained as described previously [Nallamsetty, S. and Waugh, D. S. (2007) A generic protocol for the expression and purification of recombinant proteins in Escherichia coli using a combinatorial His6-maltose binding protein fusion tag. Nat. Protoc. 2, 383-391]. The scFv was fractionated from the initial fusion by binding the His-tagged proteins (thioredoxin and TEV protease) to 1 ml Histrap HP columns (GE Healthcare). A Superdex-75 gel-filtration chromatography (Hiload 26/60, GE Healthcare) in PBS (pH 7.4) at a flow rate of 2 ml/min was used to both completely purify and to assess the degree of dimerization of the isolated scFv-h3D6. Finally, cationic exchange chromatography was implemented to completely fractionate the native state from scrambled forms of the scFv-h3D6. In order to perform functional assays {i.e. MTT [3-(4,5-dimethylthiazol-2-yl)-2,5-diphenyl-2H-tetrazolium bromide] assays}, lipopolysaccharides were removed from the protein by using Detoxi-Gel Endotoxin Removing columns (Thermo Scientific). Lipopolysaccharides are the major endotoxins of Gram-negative bacteria, and they could elicit a cellular response (up to 300% of viability in the MTT assay with SHSY-5Y neuroblastoma cell-line was detected; results not shown) in assays with cells or animals, or in prevention and/or treatment of a disease in a patient. The buffer was changed to PBS using PD-10 Desalting Columns (GE Healthcare), and protein aliquots (500 μl) were stored at −20° C. until use. The molecular mass of the different batches of purified protein was confirmed by MALDI-TOF (matrix-assisted laser-desorption ionization-time-of-flight) spectrometry. Mutagenesis was performed by QuikChange™.

### 2. Secondary Structure Determination by CD.

Protein secondary structure was monitored at different urea concentrations in PBS by far-UV CD spectroscopy from 260-200 nm in a Jasco J-715 spectrophotopolarimeter. The protein concentration was 20 μM, and 20 scans were recorded at 50 nm·min⁻¹ (response 2 sec) in 0.2 cm pathlength cuvettes.

### 3. Secondary Structure Determination by FTIR.

Samples at 100 μM in PBS were analyzed on a Variant Resolutions Pro spectrometer using a peltier mount and excavated cells with a 50-μm path (Reflex Analytical co.). Spectra were acquired at 25° C., 50° C., 60° C. and after returning to 25° C. The samples were equilibrated for 5 min at the indicated temperature before recording. Recording and data treatment were as previously reported in Biochem. J. 2011; 437:25-34.

### 4. Thermal Denaturation.

Thermal denaturation was performed at protein concentration 20 μM and a rate of 1° C. min⁻¹, and followed by CD at 218 nm or Trp-fluorescence emission at 338 nm.

### 5. Chemical Denaturation.

Chemical denaturation was performed by creating four series of progressive urea concentrations, where protein was added at 2 μM. Samples were equilibrated overnight at room temperature before being tested for Trp-fluorescence at 25° C. Excitation was performed at 290 nm and emission spectrum recorded from 310 nm to 400 nm. Both slits were set to 5, and 10 scans were averaged in a Varian Cary Elipse fluorimeter. The maximum of each emission spectrum was obtained by fitting to a three-parametric polynomial according to Protein Eng. 1995; 8:81-89. Urea concentrations were calculated with a hand refractometer according to the method given in Protein Structure, A Practical Approach (Creighton, T.E. ed.), 1st edition. 1997:383. The equilibrium parameters for denaturation were calculated for each denaturant concentration ([D]) using Equation 1:

f=F_{N}+(F_{I}*k1/(1+k1+k1*k2))+(F_{U}*k1*k2/(1+k1+k1*k2)) Equation 1

where: F_{N}=a+(b*x); F_{I}=c+(d*x); F_{U}=e+(h*x); k1=exp(−ΔG1/(R*T)); k2=exp(−ΔG2/(R*T)); ΔG1=G1−m1*x; ΔG2=G2−m2*x;
and in which the dependence of the intrinsic fluorescence in the native (F_{N}), intermediate (F_{I}) and unfolded (F_{U}) states upon increasing denaturant concentration (x) is taken into account by the terms b*x, d*x, and h*x, respectively (linear approximation). With this kind of analysis, a three-state model for the chemical denaturation is being assumed, with one intermediate species significantly accumulating apart from the native and unfolded forms of the protein.

To compare, two separated two-state fittings, ranging 0-5.5 M and 5-9.2 M urea, were performed using Equation 2 [cf. in Protein Structure, A Practical Approach (Creighton, T.E. ed.), 1st edition, 1997:383; and Methods Enzymol. 1986; 131:266-280]:

f=F_{N}+F_{U}*k/(1+k) Equation 2
where: F_{N}=a+(b*x); F_{U}=c+(d*x); k=exp(−ΔG/(R*T); ΔG=G−m*x

### 6. Limited Protease Digestion Studies.

To characterize the equilibrium intermediate state present in the chemical denaturation curves, limited thermolysin digestion was performed in urea (cf. Biochemistry 1998; 37:13120-13127). Thermolysin hydrolyzes peptide bonds on the N-terminal side of Val, Leu, Ile and Phe residues (cf. Methods Enzymol. 1977; 47:175-189). A ratio of thermolysin to scFv of 1:200 (w:w) was used in samples at 1 mg/ml of scFv-h3D6 in either 3 M or 5 M Urea, 20 mM Tris-HCl, 10 mM CaCl₂ (pH 7.8), the time-course of the digestion at 37° C. was followed for 32 h, and the samples were stopped with SDS-loading buffer. Subsequent SDS-PAGE (18%), blotting and N-terminal sequencing of the main-accumulated band were performed.

7. Three-Dimensional Model of the WT scFv-h3D6.

ScFv-h3D6 consists of the V_{H} domain linked to the V_{L} domain by a (Gly₄Ser)₃ linker. Homologs with known structure among the sequences in the PDB for each domain were searched for using BLAST (Basic Local Alignment Search Tool, cf. J. Mol. Biol. 1990; 215:403-410). Low-complexity regions of each non-redundant aligned sequence were filtered to discard false results. The PDB code of the sequence with the highest score coincident in the search of both domains was selected among a total of 24 candidates. The crystal structure of a scFv antibody against the SARS-spike protein-receptor binding-domain (with PDB code: 2GHW-B), matching the alignment with a 70% identity (94% similarity) and E-value 2e-84, was selected to construct a 3D model for the scFv-h3D6 using MODELLER 9v2 according to J. Mol. Biol. 1993; 234:779-815. Five possible conformations were initially constructed for the V_{H} and V_{L} domains. Although the linker region is the same in both scFv molecules, (Gly₄Ser)₃, a defined diffraction pattern in the template structure (2GHW-B) could not be obtained because of the linker's high degree of flexibility, and the coordinates for the model were calculated using the loop-refinement and energy minimization approach (MODELLER 9v2, cf. J. Mol. Biol. 1993; 234:779-815). Ten different structures matched the five initial models for the domains and the best conformation with minimum energy was selected using the criterion of knowledge-based potentials of ProSa2003 according to Nucleic Acids Res. 2007; 35:W407-W410.

### 8. TEM

To characterize the worm-like fibrils using an alternative method to FTIR, Aβ₁₋₄₂ peptide and a scFv were combined at the same concentration and incubated for aggregation. The samples were diluted 1:10 in PBS and quickly adsorbed on to glow-discharged carbon-coated grids. The material was stained using the method of uranyl acetate described in Proc. Natl. Acad. Sci. U.S.A. 2002; 99:16052-16057 and the samples were visualized with a Hitachi H-7000 microscope. Amyloid and worm-like fibrils populate different aggregation pathways: amyloid fibrils are straight and long, and form following a nucleated-dependent kinetics, whereas worm-like fibrils are curved and short, and form following non-nucleation-dependent kinetics.

B) Secondary Structure, Unfolding Pathway, and Stability of scFv-h3D6.

In agreement with the results previously reported in Biochem. J. 2011; 437:25-34, the FTIR spectrum of scFv-h3D6 decomposes in 64% native β-sheet component, 22% loops/turns component, 11% β-turns components, and 3% of a low-frequency component (see later).

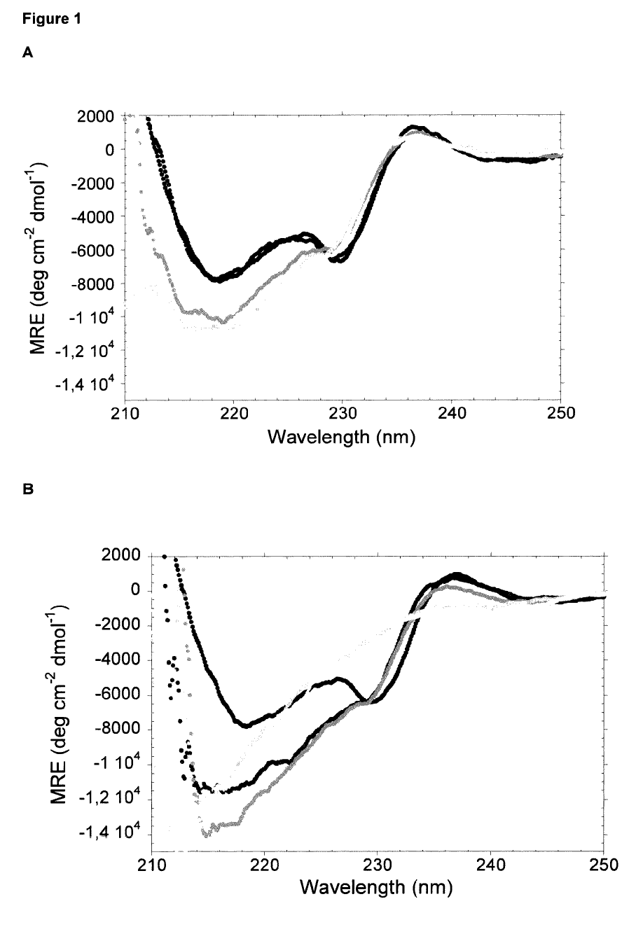

The far-UV circular dichroism (CD)-spectrum of the native scFv-h3D6 showed an ellipticity minimum at 218 nm and an ellipticity maximum at 200 nm, as expected from an all β-sheet fold (FIG. 1), but a second ellipticity minimum at 230 nm and a positive shoulder at 237 nm were found (cf. Biochem. J. 2011; 437:25-34). These anomalies are contributions from the aromatic or cystinyl side-chains within the far-UV (cf. Biochemistry 1999; 38:10814-10822). The minimum at 230 nm was also reported for an IgG1-Fc (cf. Biochim. Biophys. Acta 2012; 1824:542-549) and described for some V_{L} domains (cf. Protein Eng. Des. Sel. 2007; 20:481-490 and J. Biol. Chem. 2008; 283:15853-15860), the latter attributed to the interaction of the aromatic residues with the conserved Trp35. The positive shoulder at 237 nm is due to the conserved Trp36 in V_{H} domain, since it is conserved upon mutation of the onlu disulphide bridge of the domain [Montull-Gaya, Laia, Master's experimental work 2013: Análisi de les conseqüéncies termodinámiques i conformacionals d'eliminar el pont disulfur del domini V_{H} d'un fragment d'anticós anti-abeta (scFv-h3D6)].

Because the high-yield production of scFvs is limited by their folding and stability properties, the unfolding pathway of scFv-h3D6 was examined. In contrast to thermal denaturation (cf. Biochem. J. 2011; 437:25-34), urea denaturation led to the unfolding of the molecule and not to aggregation. The unfolding followed a three-state transition, as is reported for other scFv molecules (cf. Biochemistry 1998; 37:13120-13127; J. Mol. Biol. 1999; 290:535-546; FEBS Lett. 1999; 462:307-312). When denaturation of scFv-h3D6 by urea was followed by CD (FIG. 1), the initial ellipticity minimum at 230 nm was progressively lost as the concentration of urea was increased from 2 M to 3.7 M urea, while the ellipticity minimum at 218 nm accentuated and blue-shifted to 215 nm (FIG. 1A). The ellipticity maximum at 237 nm was lost between 6.9 M and 8 M urea (FIG. 1B), but a pure random-coil conformation was not achieved even at 8.6 M urea. This result indicated the presence of some residual secondary structure in the urea-denatured state. After dialyzing from the different urea concentrations to PBS, the initial spectrum was completely recovered, revealing that the chemical unfolding of scFv-h3D6 was 100% reversible (data not shown).

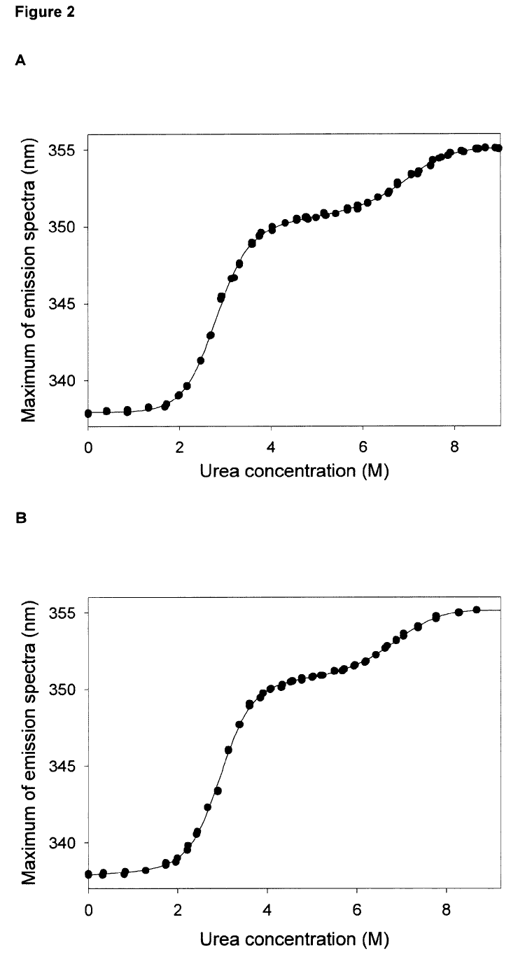

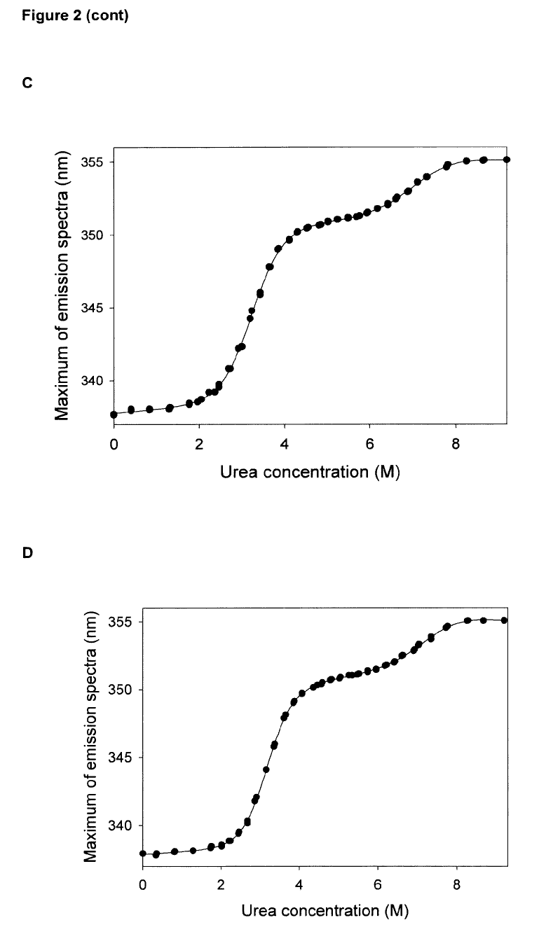

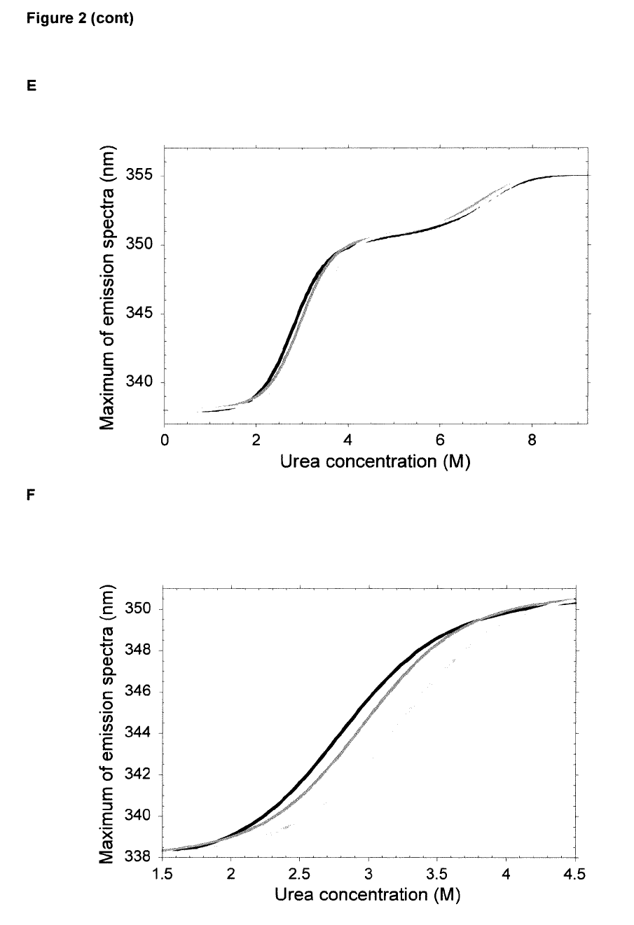

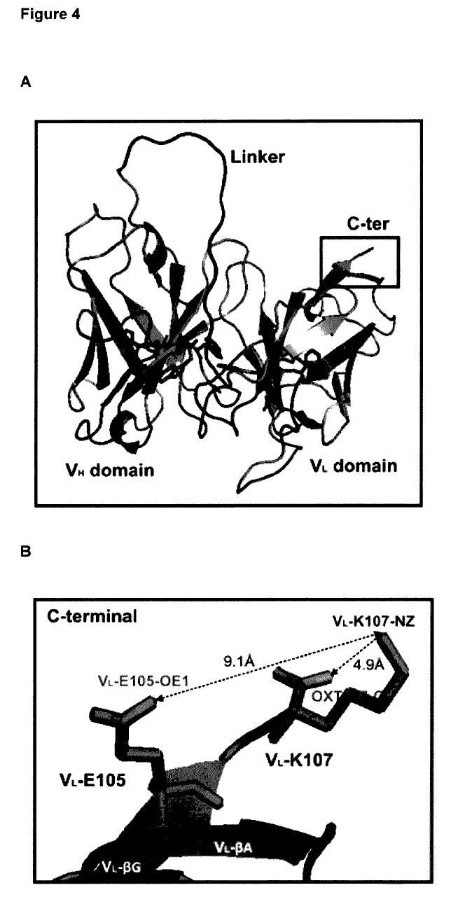

Chemical denaturation allows for the determination of the thermodynamic parameters in the unfolding process by plotting discrete points in equilibrium (FIG. 2). Trp-fluorescence is the best choice to follow unfolding processes because Trp residues are typically buried in hydrophobic cores of proteins and both the intensity and the location of the maximum of the emission spectrum change upon exposition to the solvent. ScFv-h3D6 contains five buried Trp residues, one within the core of each domain (VH-W36, VL-W35) and three in the interface between both domains (VH-W47, VH-W103, VL-W89; Kabat numbering, cf. Kabat E. A., Wu T. T., Reid-Miller M., Perry H., and Gottesman K., eds. Sequences of Proteins of Immunological Interest. Fourth Edition ed. US Govt. Printing Off. No. 165-492; 1987) (FIG. 4). These residues in the interface became completely exposed when the less-stable domain unfolded, thereby generating an intermediate state that was populated at ˜5 M urea. Pluckthun and co-workers observed that, because three processes occur in the unfolding of a scFv molecule (interface disruption, denaturation of one domain, denaturation of the other domain), the changes in the intensity of the spectra can increase or decrease, and may mutually compensate each other. Thus, because the changes in the intensity of the spectra can increase or decrease upon unfolding when more than one Trp residue is present, as is the case for scFv-h3D6, they may mutually compensate each other and, therefore, this determination would make no sense. This effect does not occur when analyzing data for emission maximum because the shift is always in the same direction, and thus the data for the red-shift reliably show the exposition of the Trp-residues to the solvent. Therefore, the proper way to quantify Trp-fluorescence changes in the unfolding of any scFv molecule is following the emission maximum of the spectra, which displays a red-shift upon exposition to the solvent (cf. Biochemistry 1999; 38:8739-8750). FIG. 2A shows that the red-shift in the fluorescence emission spectra of the wild type scFv-h3D6 (WT) consists of two transitions with plateaus at ˜351 and at 355 nm. The plateau at ˜351 nm spans the 4-6 M urea region and is indicative of the occurrence of an intermediate state. The final plateau reaches the emission maximum considered for tryptophan-containing fully-unfolded proteins, 355 nm [cf. the disclosure in Protein Structure: A Practical Approach (Creighton, T.E., ed.). 2nd edition, 1997:261-267] and therefore indicates that the tertiary structure is completely disrupted upon urea denaturation.

Pluckthun and co-workers have proposed a model where, depending on the stability of the domains' interface and on the intrinsic stability of each domain, unfolding of scFv molecules may be sequential (cf. Biochemistry 1998; 37:13120-13127; J. Mol. Biol. 2001; 305:989-1010). This model classifies the scFv fragments into four classes and, among them, Class I is characterized by the intrinsic stability of one domain being significantly higher than the total stability (intrinsic plus interface contribution) of the other domain. In consequence, an unfolding intermediate state would accumulate in the equilibrium and a clear step in the unfolding curve would be detected. This step in the fluorescence emission maximum plot will be more evident when the first domain to unfold is V_{L} because it will completely disrupt the interface, exposing the V_{H} Trp residues. This is the case for scFv-h3D6, which shows 37 KJmol⁻¹ for the intrinsic stability of the V_{H} domain and 19 KJmol⁻¹ for the sum of the intrinsic stability of the V_{L} domain plus interface contribution.

The first transition corresponds to the unfolding of the less-stable domain (although interface disruption may also contribute to the unfolding process), whereas the second transition corresponds to the unfolding of the more-stable domain from the intermediate state. The parameters for both transitions are similar when fitted to a two-state model (from 0 to 5.5 M urea and from 5 to 9.2 M). In summary, the free energy of unfolding in water of the less-stable domain (ΔG_{N-I}) of the WT form, together with interface disruption, is −19.3±0.7 KJmol⁻¹. The m_{I-N} value, which reflects the difference in solvent accessibility between the intermediate state and the native state, is 7.0±0.3 KJmol⁻¹M⁻¹ (Table 1). This value is similar to the m values published for correctly folded domains of similar sizes (cf. Protein Sci. 1995; 4:2138-2148). The thermodynamic stability of the more-stable domain (ΔG_{I-U}) is −36.9±5.7 KJmol⁻¹ and the m_{U-I} value is 5.2±0.8 KJmol⁻¹M⁻¹(Table 1). The resulting [D]_{50%} are 2.8 M and 7.1 M, respectively.

The fitting to the three-state model of the plot for emission maximum is shown in Table 1.

TABLE 1
Fitting to three-state model of equilibrium denaturation curves of the WT scFV-
h3D6 and its elongation mutants. Fitting to the two-state model has been done for
0-5.5M Urea and for 5-9.2M Urea, to be compared with the three-state model.

| Parameter | First transition | | | Second transition | | |
|---|---|---|---|---|---|---|
| Variant, states | ΔG_{I-N} | m_{I-N} | [D]_{I-N} | ΔG_{U-I} | m_{U-I} | [D]_{U-I} |
| WT, three-state, | 19.3 ± 0.7 | 7.0 ± 0.3 | 2.76 | 36.9 ± 5.7 | 5.2 ± 0.8 | 7.10 |
| two indep. two-state | 19.7 ± 0.7 | 7.1 ± 0.3 | 2.78 | 32.0 ± 4.2 | 4.5 ± 0.6 | 7.11 |
| C1, three-state, | 21.0 ± 0.9 | 7.1 ± 0.3 | 2.96 | 31.1 ± 6.4 | 4.5 ± 0.9 | 6.91 |
| two indep. two-state | 21.1 ± 1.0 | 7.2 ± 0.4 | 2.93 | 35.7 ± 3.7 | 5.1 ± 0.5 | 7.00 |
| C2, three-state, | 22.3 ± 0.7 | 6.9 ± 0.2 | 3.23 | 36.2 ± 5.8 | 5.1 ± 0.8 | 7.10 |
| two indep. two-state | 22.5 ± 0.8 | 7.0 ± 0.3 | 3.21 | 37.7 ± 3.6 | 5.3 ± 0.5 | 7.11 |
| C3, three-state, | 24.4 ± 0.5 | 7.7 ± 0.2 | 3.17 | 38.6 ± 3.9 | 5.3 ± 0.6 | 7.28 |
| two indep. two-state | 24.6 ± 0.5 | 7.8 ± 0.2 | 3.15 | 36.5 ± 4.8 | 5.0 ± 0.6 | 7.30 |

ΔG in KJ mol⁻¹;
m in KJ mol⁻¹ M⁻¹.
N = native state;
I = intermediate state;
U = unfolded state;
[D] between states = denaturant 50% in M.

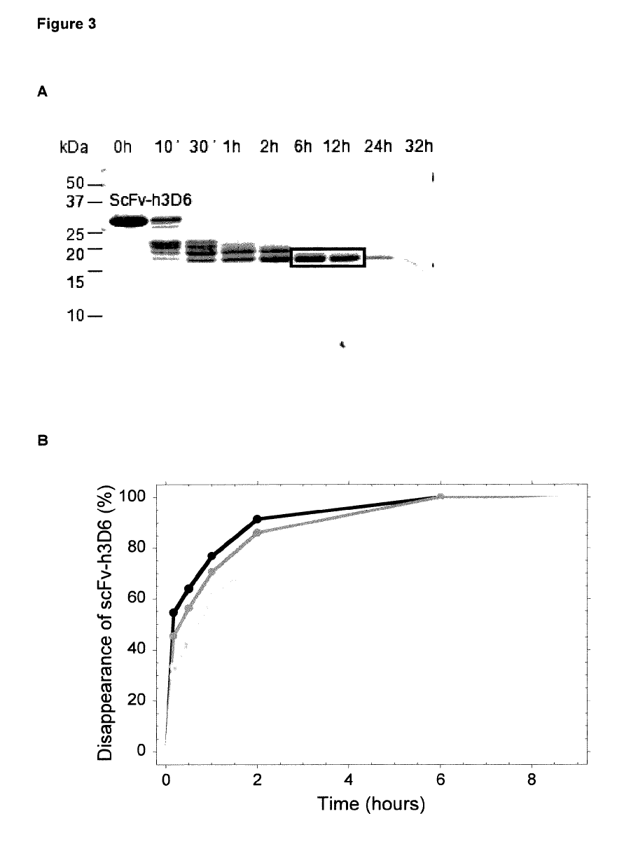

The assignation of each domain to the transitions in the unfolding has been corroborated by limited proteolysis of the intermediate state and subsequent N-terminal sequencing of the proteolysis-resistant domain (V_{H}). As such, to characterize the equilibrium intermediate, limited proteolysis with thermolysin was performed at 5 M urea at 37° C. (FIG. 3A). After 1 h of proteolysis, a 17 kDa main-band accumulated: this band was blotted and N-terminal sequenced, rendering the sequence of the V_{H} domain (GAMEVQ). Thus, the proteolysis-resistant band must consist of the V_{H} domain (13.13 kDa) plus the linker region (0.96 kDa) and a small part of the V_{L} domain (3.01 kDa; FR1 and half of CDR1, plausibly ending at V_{L}-L27B, Kabat numbering, cf. J. Exp. Med. 1970; 132:211-250; and Nucleic Acids Res. 2001; 29:205-206). The resistance to proteolysis of V_{L} β-strands A and B in the intermediate suggests the maintenance of the long and stable β-hairpin that they form in the native state (not shown). Also in consonance with the proteolysis of the intermediate state and subsequent N-terminal sequencing of the proteolysis-resistant domain, the initial ellipticity minimum at 230 nm in the CD spectra, characteristic of some V_{L} domains (cf. Protein Eng. Des. Sel. 2007; 20:481-90; J. Biol. Chem. 2008; 283:15853-60) progressively disappears during the first transition. In conclusion, chemically-induced unfolding is characterized by the presence of an intermediate state, whereby the intermediate state in the urea denaturation curves consists of the unfolded V_{L} domain and the folded V_{H} domain, suggesting the V_{L} domain of scFv-h3D6 as a target for thermodynamic stability (i.e. protein stability) redesign. To this end, the 3D-structure of scFv-h3D6 was modeled.

### C) Three-Dimensional Model and Redesign of the WT Fold.

Ig (antibody) variable domains contain two antiparallel β-sheets packed tightly against each other in a compressed β-barrel (FIG. 4). The N- and C-terminal end of each domain are located apart at the top and bottom of the β-barrel. One of the sheets contains five strands (A, B, C, C ‘, C″, the additional C’ and C″ are about constant region domains) and the other sheet contains four strands (D, E, F, G). The complementary-determining regions (CDRs) are loops located in between B-C, C′-C″, and F-G β-strands, and cluster at one end of the β-barrel. The fold is stabilized by hydrogen bonding between β-strands of each sheet, hydrophobic interaction between residues of opposite β-sheets, and a disulfide bridge between two β-sheets (strands B and F, C22-C92 in V_{H} and C23-C88 in V_{L}; Kabat numbering, cf. J. Exp. Med. 1970; 132:211-250; and Nucleic Acids Res. 2001; 29:205-206).

ScFv-h3D6 contains five tryptophan residues (Figures and 9, red), three in the V_{H} domain (the conserved W36, W47 and W103, in strands C, C′, and G, respectively) and two in the V_{L} domain (the conserved W35 in strand C and W89, the first residue of CDR3, in strand F). V_{H}-W36 and V_{L}-W35 constitute the center of the hydrophobic core in each domain, whereas V_{H}-W47, V_{H}-W103, and V_{L}-W89, are located in the domains' interface. As a point of reference, and an indication of the relevance of hydrophobic interactions in this interface, the distance between V_{H}-W103-CZ2 and V_{L}-W89-CH2 is 3.3 Å, and that between V_{H}-W47-CD1 and V_{L}-W89-CZ2 is 5.7 Å.

Using graphical examination of the model structure, the V_{L} domain, located at the C-terminal end of the molecule, was observed to end before its latest β-strand was completed. It was additionally found that the C-terminal end of the molecule, which corresponds to V_{L}-K107, still belongs to β-strand G (FIG. 4, inset). Therefore, the side-chains of V_{L}-K107 and V_{L}-E105 are faced to the same side of the β-strand and should establish an electrostatic interaction. This electrostatic interaction is not properly performed since the distance between V_{L}-K107-NZ and V_{L}-E105-OE1 is 9.1 Å. This interaction might be weakened by the attraction of the V_{L}-K107-NZ to the OXT107-O (i.e. by the proximity of the OXT107-O). The distance between V_{L}-K107-NZ and the OXT107-O is 4.9 Å, which means that the side-chain of V_{L}-K107 is bent towards the OXT107-O. Hence, elongation of the C-terminal end should separate the OXT-O from the side-chain of V_{L}-K107 and propitiate secondary structure stabilization. To test the possibility of the intrinsic stability of the V_{L} domain being increased by elongation of its C-terminal end, the C-terminal residue of the twenty-four scFv molecules with similar sequence used in the structural alignment was examined [see A in FIG. 4]. Material and methods, above], whereby the structural alignment showed that 16 of them have as the C-terminal V_{L}-K107, 6 V_{L}-R108 and 2 V_{L}-T109 (i.e. 16 finished at V_{L}-K107, six at V_{L}-R108 and two at V_{L}-T109 residues). Accordingly, in an attempt to stabilize the end of the β-strand G, it was elongated one or two residues to generate the elongation mutant forms V_{L}-el-R108 and V_{L}-el-R108T109, respectively. In addition, and to test the hypothesis on the above mentioned effect of OXT107-0 on weakening the electrostatic interaction between V_{L}-K107 and VL-E105 (i.e. on β-strand G packing), the main-chain was extended by just adding a Gly residue in elongation mutant V_{L}-el-R108G. For the sake of clarity, these are herein referred to as C1 (V_{L}-el-R108G), C2 (V_{L}-el-R108) and C3 (V_{L}-el-R108T109).

### D) Secondary Structure, Unfolding Pathway, and Stability of the Elongation Mutants.

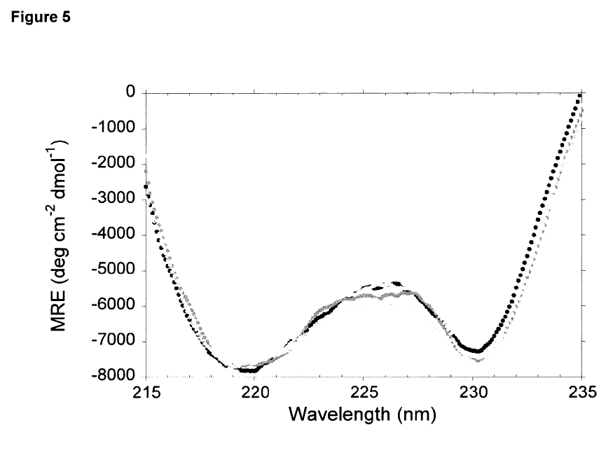

As expected, the CD spectra of the elongation mutants are slightly different to that of the native protein. In fact, mutation induces a more marked minimum at 230 nm (FIG. 5), plausibly indicating a more populated VL native-state upon stabilization.

FIGS. 2B-2D show the urea denaturation curves of the elongation mutants as a function of the red-shift in the fluorescence emission spectra. Compared to the WT curve (FIGS. 2E-2F), the unfolding of the V_{L} domain is similar for C1, whereas higher urea concentrations are required for C2 and C3. These forms are thus more stable than the WT scFv-h3D6. The fitting of these data (Table 1) shows that thermodynamic stability of the elongation mutants improves in the order C1 (ΔΔG≈1.7 KJmol⁻¹), C2 (ΔΔG≈3.0 KJmol⁻¹) and C3 (ΔΔG≈5.1 KJmol⁻¹). Although the thermodynamic stability for the mutant elongated just with a Gly (C1) is only slightly increased (i.e. the difference is small ≈1.7 KJmol⁻¹), stabilization concurs with the fact that the aggregation tendency has been diminished as a consequence of the stabilization of the native state. Cooperativity (m_{I-N} value) is maintained in C1 and C2, and as a consequence [D]_{50%} increases with thermodynamic stability (0.2 M and 0.5 M, respectively). In contrast, the most stable elongation mutant, C3, has an increased cooperativity that results in an increase [D]_{50%} of just 0.5 M (similar to that of C2). Thus, cooperativity is maintained in elongation mutants extended by one residue, but increased in the case of the C3 elongation mutant. This increase in cooperativity indicates a more compact native state upon mutation. In the case of the V_{H} domain, determination of the equilibrium parameters is delicate because the red-shift of its transition is small (3-4 nm), i.e., SD is much higher than in the first transition; however, no differences are likely to exist among elongation mutants.

It was questioned whether these increases in stability would be reflected in the kinetics of thermolysin proteolysis in urea. As expected, at urea concentrations where the intermediate state populates all of the elongation mutants (i.e. 5 M), the kinetics is exactly that depictured for the WT in FIG. 3A (not shown). At the mid-point of the first transition (i.e. 3 M), however, stabilization should decrease the population of the intermediate state. In full agreement, the disappearance of the initial band delays as the thermodynamic stability of the different elongation mutants increases (FIG. 3B).

### D) Changes in the Heat-Induced Aggregation Pathway Upon Elongation of the C-Terminal End.

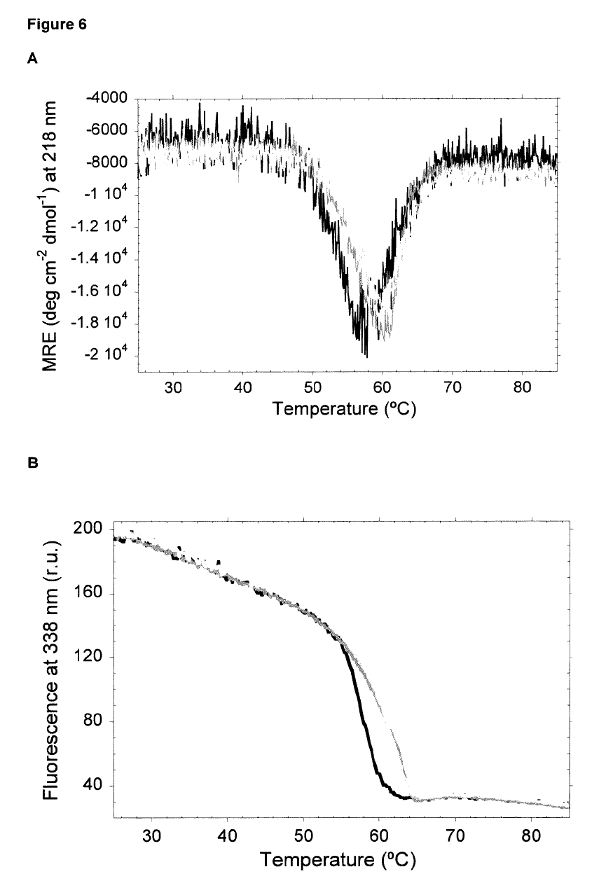

The irreversible aggregation pathway of scFv-h3D6 is already reported and is featured by an intermediate state different from that populating the unfolding pathway (cf. Biochem. J. 2011; 437:25-34). Among other differences, thermolysin proteolysis showed that both domains of the heat-induced intermediate are equally susceptible to digestion. This intermediate is more abundant at ˜60° C., and thermal denaturation followed by CD or fluorescence (Trp-fluorescence) shows a conformational reorganization from the WT native state starting beyond 50° C. (i.e. beginning beyond 50° C. and finishing at ˜60° C., FIG. 6). This conformational reorganization (transition) is shifted to higher temperatures in all three elongation mutants, indicating a diminished tendency toward aggregation upon extension of the C-terminal end of the scFv by one or two residues.

The WT scFv-h3D6 was reported to aggregate in the form of worm-like fibrils (WL), as shown by the visualization of curved fibrils by transmission electron microscopy (TEM) and by the appearance of a FTIR component centered at 1626 nm (cf. Biochem. J. 2011; 437:25-34).

To study the worm-like (WL) component in the different elongation mutants, FTIR spectra were acquired at different temperatures (Table 2). Analysis of the IR amide I′ band at different temperatures allows deepening in the aggregation process because it has the capability of distinguishing among different β-sheet structures. The amide I′ band arises from the C═O stretching vibration of each peptidic bond and the strength of coupling depends greatly on the conformation of the polypeptide backbone. As a general rule, the position of β-sheet components shifts to lower wavenumbers as a result of increased hydrogen bonding, a more planar sheet, or a larger number of strands (cf. Protein Sci. 2004; 13:3314-3321). At 25° C., there are no observable differences among elongation mutants in the native β-sheet component (1637 cm⁻¹), which comprises ˜65% of the area of the spectrum. The rest of the spectrum corresponds to loops/turns (1700-1660 cm⁻¹) and is also similar among the elongation mutants. This is not surprising since FTIR is a low-resolution spectroscopy and data treatment can mask small differences in secondary structure. At the temperature where the reorganization of the native state begins (50° C.), the secondary structure is similar to the native form for all of the elongation mutants. In fact, there are no differences among WT and elongation mutants spectra before thermal transition (25° C. and 50° C.), being dominated by the native β-sheet component (1637 cm⁻¹). In contrast, once the reorganization has led to the intermediate state (60° C.), differences in aggregation tendency are reflected in the distribution of the FTIR components corresponding to β-structures and more compact β-sheet components (centered at lower frequencies) appear.

TABLE 2
FTIR analysis of the WT scFV-h3D6 and its elongation mutants at different
temperatures.

| | Center | Area | Center | Area | Center | Area | Center | Area |
|---|---|---|---|---|---|---|---|---|
| | (cm⁻¹) | (%) | (cm⁻¹) | (%) | (cm⁻¹) | (%) | (cm⁻¹) | (%) |
| | WT 25° C. | | WT 50° C. | | WT 60° C. | | WT Renat | |
| High-freq. antipar. β-sheet | 1688 | 2 | 1689 | 2 | 1685 | 3 | 1685 | 2 |
| β-turns | 1674 | 9 | 1677 | 8 | 1676 | 3 | 1672 | 5 |
| Loops/turns | 1659 | 22 | 1662 | 22 | 1661 | 30 | 1662 | 17 |
| α-helix | | | | | 1650 | 7 | 1653 | 10 |
| Random coil | | | | | 1643 | 7 | 1645 | 6 |
| Native β-sheet | 1637 | 64 | 1637 | 65 | 1636 | 16 | 1637 | 19 |
| WL β-sheet | | | | | 1627 | 23 | 1626 | 26 |
| Amyloid β-sheet | | | | | 1616 | 10 | 1616 | 14 |
| Side-chains | 1614^{a} | 3 | 1613^{a} | 3 | 1607 | 1 | 1607 | 1 |
| | C1 25° C. | | C1 50° C. | | C1 60° C. | | C1 Renat | |
| High-freq. antipar. β-sheet | 1685 | 3 | 1689 | 1 | 1685 | 0 | 1685 | 2 |
| β-turns | | | 1675 | 13 | 1678 | 9 | 1679 | 8 |
| Loops/turns | 1663 | 30 | 1659 | 21 | 1662 | 16 | 1667 | 14 |
| α-helix | | | | | 1653 | 13 | 1655 | 10 |
| Random coil | | | | | 1643 | 18 | 1643 | 19 |
| Native β-sheet | 1637 | 66 | 1637 | 63 | 1632 | 23 | 1632 | 20 |
| WL β-sheet | | | | | 1623 | 15 | 1621 | 18 |
| Amyloid β-sheet | | | | | 1614 | 5 | 1614 | 9 |
| Side-chains | 1611 | 1 | 1614^{a} | 2 | 1607 | 1 | 1607 | 2 |
| | C2 25° C. | | C2 50° C. | | C2 60° C. | | C2 Renat | |
| High-freq. antipar. β-sheet | 1688 | 2 | 1683 | 4 | 1683 | 3 | 1681 | 5 |
| β-turns | 1678 | 5 | 1675 | 0 | 1671 | 14 | 1669 | 13 |
| Loops/turns | 1662 | 26 | 1663 | 30 | 1662 | 5 | 1659 | 8 |
| α-helix | | | | | 1652 | 20 | 1652 | 15 |
| Random coil | | | | | 1643 | 12 | 1643 | 16 |
| Native β-sheet | 1637 | 66 | 1638 | 65 | 1636 | 15 | 1631 | 23 |
| WL β-sheet | | | | | 1627 | 18 | 1621 | 15 |
| Amyloid β-sheet | | | | | 1617 | 11 | 1614 | 5 |
| Side-chains | 1612^{a} | 1 | 1613^{a} | 1 | 1607 | 1 | 1607 | 1 |
| | C3 25° C. | | C3 50° C. | | C3 60° C. | | C3 Renat | |
| High-freq. antipar. β-sheet | 1689 | 2 | 1689 | 2 | 1685 | 4 | 1689 | 1 |
| β-turns | 1677 | 9 | 1678 | 10 | 1676 | 7 | 1679 | 9 |
| Loops/turns | 1661 | 22 | 1661 | 23 | 1666 | 6 | 1663 | 18 |
| | | | | | 1657 | 23 | 1657 | 2 |
| α-helix | | | | | 1650 | 5 | 1648 | 17 |
| Random coil | | | | | 1640 | 18 | 1640 | 5 |
| Native β-sheet | 1637 | 65 | 1637 | 63 | 1630^{b} | 21 | 1630^{b} | 30 |
| WL β-sheet | | | | | 1619^{b} | 15 | 1619^{b} | 13 |
| Amyloid β-sheet | | | | | | | 1613^{b} | 3 |
| Side-chains | 1612^{a} | 2 | 1613^{a} | 1 | 1608 | 1 | 1608 | 2 |

^{a}this band, quantitatively negligible, could also correspond to the amyloid component.
^{b}These bands are difficult to be attributed because they are centered at boundaries.

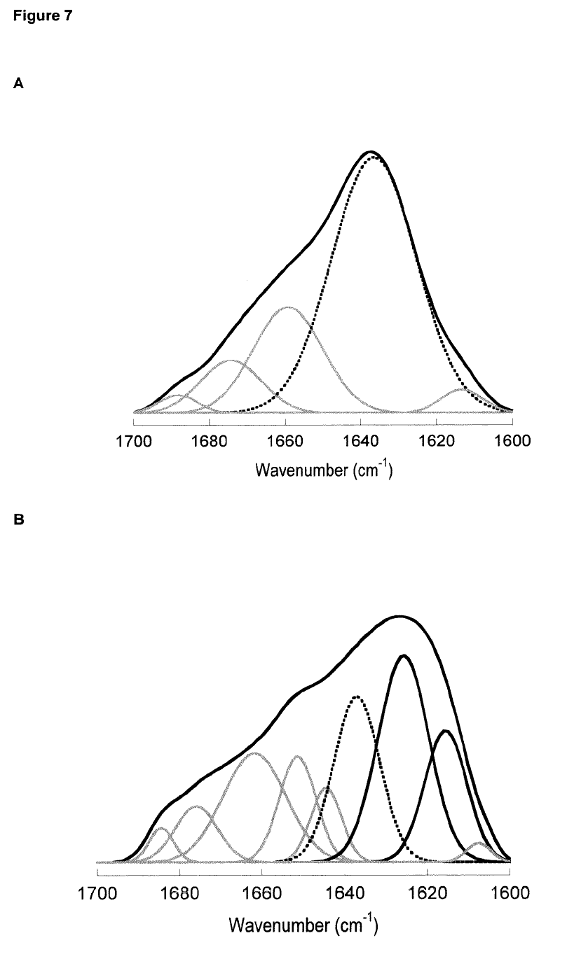

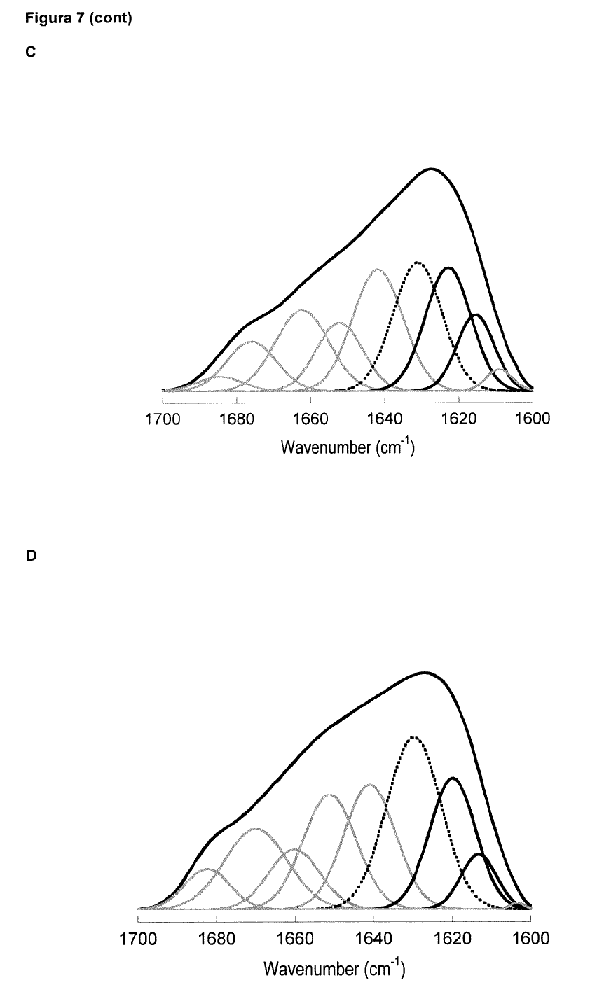

For the WT, the main conformational transition from the native state to the intermediate state implies a substantial decrease in the native β-sheet component (1637 cm⁻¹) in favor of the emergence of more compact β-sheet components (centered at lower frequencies; Table 2). For the WT, these components were previously attributed to worm-like (WL) (1627 cm⁻¹) and amyloid (1616 cm⁻¹) fibrils, and the WL component was shown to govern the spectra at 60° C. nm (cf. Biochem. J. 2011; 437:25-34). When cooling is allowed, all three β-sheet bands increase at the expense of the loop/turn component, and the WL component finally contributes 26% to the area of the spectrum (Table 2, FIGS. 7A-B).

Upon extending the C-terminal end of scFv-h3D6 progressively (from C1 to C3), more native β-sheet is kept in the intermediate state and the WL and amyloid components are diminished. In the case of C1 (Table 2, FIG. 7C), more native β-sheet is kept in the intermediate state, the WL component does not predominate, and the amyloid component is diminished with respect to the WT. The maintenance of the native β-sheet component is also observed in the renatured states of C2 (Table 2, FIG. 7D) and, especially of C3 (Table 2, FIG. 7E). It is noticeable that the wavenumbers for all three β-sheet components (i.e. the center of the bands corresponding to β-sheet structure) are shifted to lower frequencies (˜5 cm⁻¹) in the elongation mutants, which indicates a higher-density interaction-network among the side-chains. This is especially clear for the most stable elongation mutant C3 (R108T109), where it is difficult to differentiate among bands because they are at the boundary between the values previously defined in the literature for different β-structures (cf. Biochem. J. 2011; 437:25-34; Protein Sci. 2004; 13:3314-3321). Although it is tempting to correlate the different FTIR spectra of the heat-induced intermediate of elongation mutant C3 to the higher cooperativity of its V_{L} domain (m_{I-N} value), this putative correlation cannot be concluded because elongation mutants C1 and C2, which maintain the ml-N value, also showed a different FTIR spectra (despite to a lesser extent than that of C3). Thus, the intermediate state of C3 shows these shifts in a more marked manner and, more importantly, has a different conformation. Concretely, the band for the amyloid component is negligible, the WL component is diminished, and the native β-sheet component is bigger than in the WT and elongation mutants C1 and C2 (Table 2, FIG. 7E).

FTIR analysis allows the conclusion that the stabilization of the native state by extending the C-terminal end drives to a destabilization of the heat-induced intermediate state and a subsequent decrease in the aggregation tendency of the molecule, i.e. elongation of the C-terminal domain increases stability and diminishes aggregation tendency of the scFv.

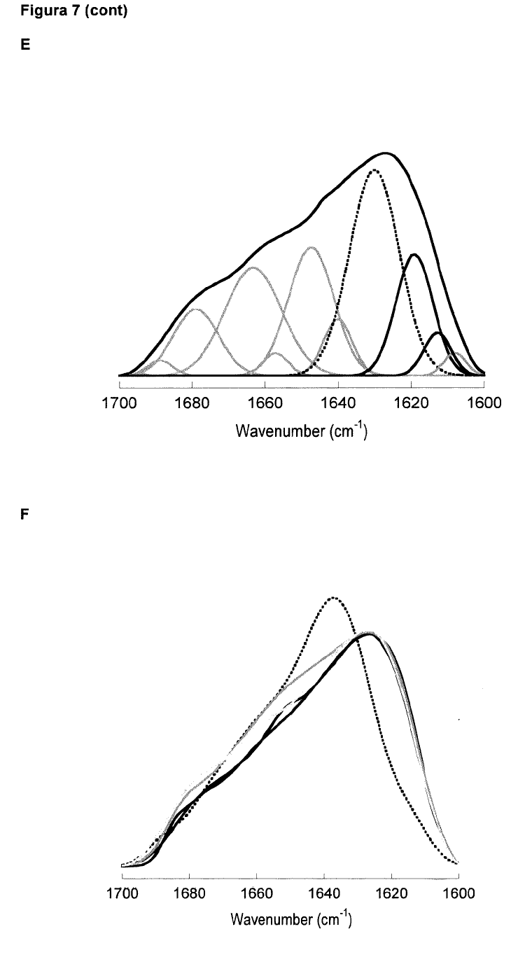

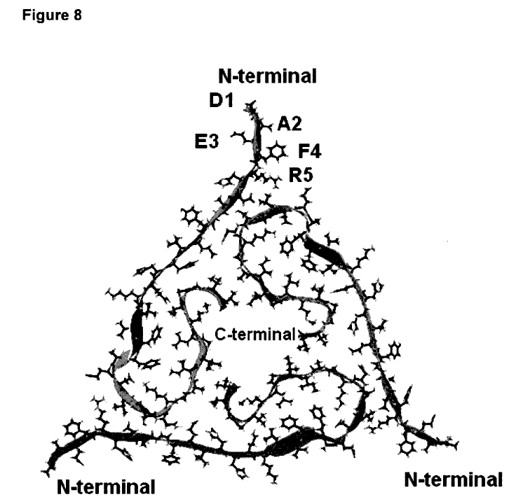

Although aggregation tendency has clearly diminished upon mutation, the WL fibril component is more or less present in all of the C-terminal extension mutants C1 to C3. It was determined using FTIR that, as described for the WT scFv-h3D6-Aβ₁₋₄₂ complex, WL fibrils, are formed from all of the elongation mutants C1 to C3. The formation of WL fibrils by the scFv-h3D6:Aβ₁₋₄₂ complex is the basis for the protective effect of scFv-h3D6 (cf. Biochem. J. 2011; 437:25-34) and thus this result is critical to the elongation mutants of the present invention. Since the antibody (Bapineuzumab) from which scFv-h3D6 derives recognizes the 1-5 N-terminal sequence of the Aβ peptide and captures it in a helical conformation (cf. Sci. Rep. 2013; 3:1302) and the three dimensional structure of the oligomers extracted from humans (cf. FIG. 8) is based on trimeric stacks leaving the N-terminal residues 1-5 (precisely the epitope recognized by scFv-h3D6) exposed to solvent (cf. Cell 2013; 154(6):1257-1268) then the wild-type and elongation mutants of the present invention recognize the most solvent-exposed regions of these oligomers.

Because clearance of Aβ oligomers by scFv-h3D6 parallels the restoration of the apoE and apoJ concentrations in vivo (3×Tg-AD mouse model), it has been proposed that those apolipoproteins are responsible for the clearance of the h3D6:Aβ complex [MAbs. 2013; 5(5): 665-677]. This concurs with the occurrence of hydrophobic residues in the surface of the h3D6:Aβ₁₋₄₂ complex, as described in Biochem. J. 2011; 437(1): 25-34. In conclusion, it was observed that the extension of the C-terminal end decreased the aggregation tendency of the isolated scFv molecules, C1 to C3, but not that of the corresponding scFv-h3D6:Aβ₁₋₄₂ complexes formed therefrom. Thus, the fold was improved without impairing its protective effect.

In summary, stabilization of the native state has resulted in destabilization of the heat-induced intermediate state and subsequent decrease in the aggregation tendency of the fold (i.e. elongation of the C-terminal domain of an anti-amyloid β single-chain variable fragment increases its thermodynamic stability and decreases its aggregation tendency). In an in vivo model, these elongation mutants, C1 to C3, with increased thermodynamic stability and diminished aggregation tendency show increased half-life and, consequently, are effective at lower doses than the WT form.

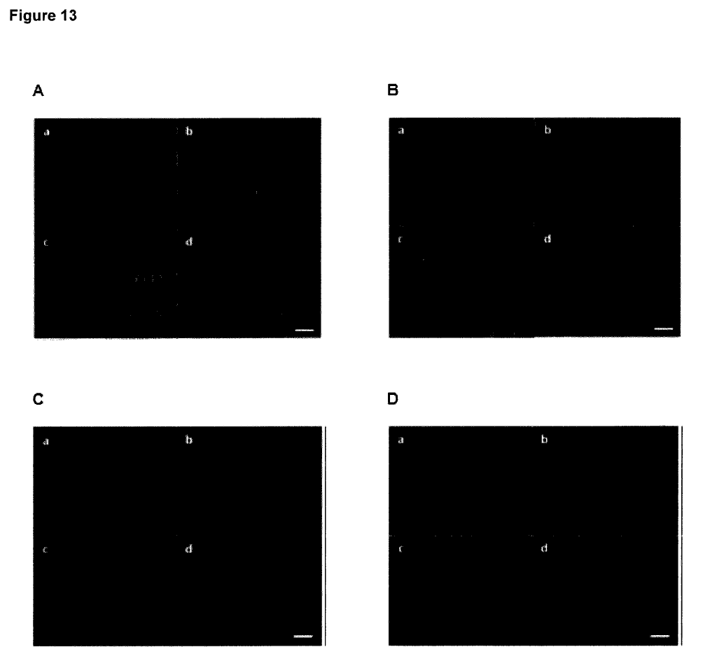

The WT scFv-h3D6 has recently been shown to be beneficial in a mouse model of Alzheimer's disease (AD) [MAbs. 2013; 5(5):665-677], whereby after a single intraperitoneal dose the high swimming navigation speed of triple-tansgenic 3×Tg-AD mice was reversed to normal levels, the short- and long-term learning and memory deficits were ameliorated. In addition, brain tissues of the treated animals revealed a global decrease of the dodecameric, nonameric, hexameric and trimeric Aβ-oligomers in the cortex and olfactory bulb, while in the hippocampus and cerebellum these remained the same (cf. FIG. 10 A, B and FIG. 13, A-D). Since Aβ oligomers are known to be the toxic species triggering Alzheimer's disease pathology, scFv-h3D6 treatment is curative.

These oligomers are multiples of 3 (trimers, hexamers, nonamers and dodecamers, cf. FIG. 8 and Cell 2013; 154(6):1257-1268), so that the effectiveness of an antibody fragment directed against the N-terminal 1-5 residues in removing those species in vivo makes sense when the recently-determined three-dimensional structure for human Aβ peptide oligomers is taken into account. This structure is based on the stacking of various trimeric units, with 0.48 nm displacements along the fibril axis, and which orients the N-terminal 1-5 residues of each momomer to the solvent.

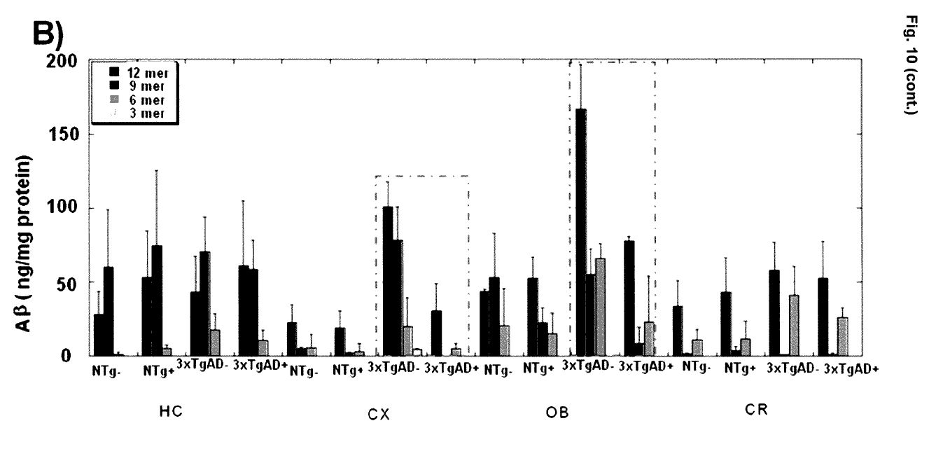

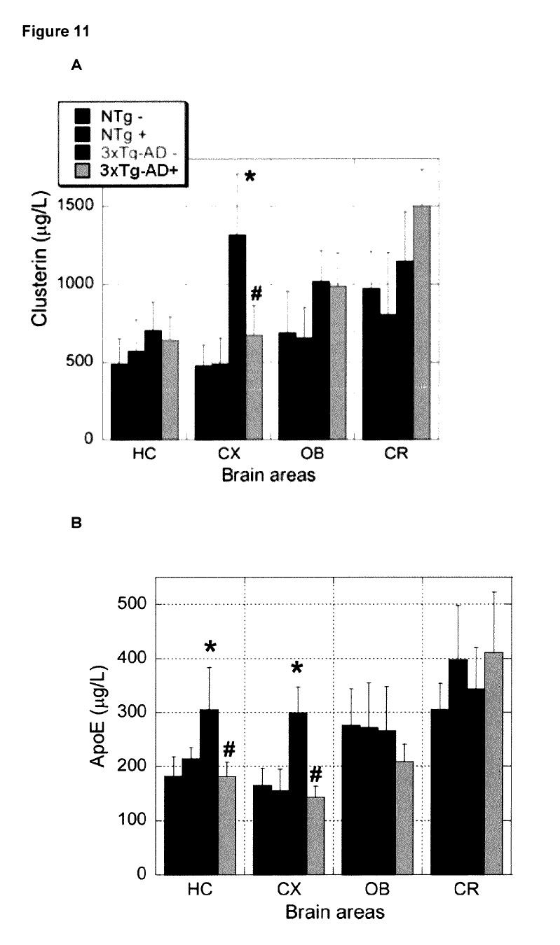

On the other hand, an increase of both clusterin (apoJ) and apoE concentrations have been described in the cortex, as well as an increase of apoE in the hippocampus, for the 3×Tg-AD animals, cf. FIG. 11 A, B. ScFv-h3D6 treatment significantly recovered the non-pathological levels of these apolipoproteins. ApoE is involved in the activation of glial cells in Alzheimer's disease pathology, thus, the recovery of the non-pathological concentration of such an apolipoprotein upon treatment indicates that inflammation has been also reduced (i.e. that the scFv of the invention does not activate neuroinflammation).

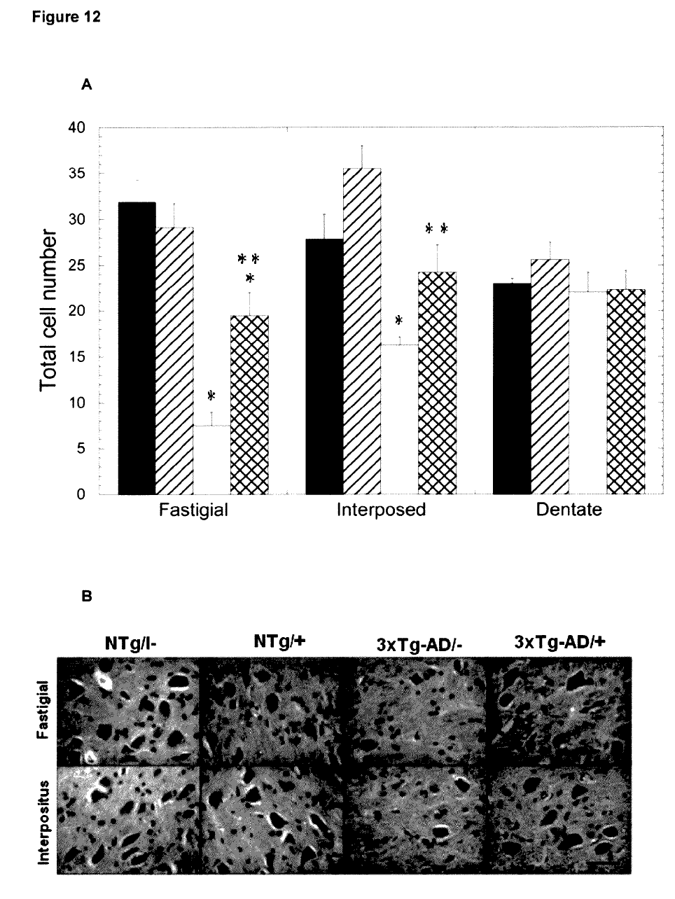

Finally, scFv-h3D6 treatment recovered the cell number in several areas of the brain of the 3×Tg-AD mouse model [cf. mAbs. 2013; 5(5):660-664] and removed intracellular Aβ-peptide as seen by immunohistochemistry (Esquerda, G. Master's Thesis, Universitat AutOnoma de Barcelona, 2013), cf. FIG. 12 A, B. In particular, neuron depletion in the DCN of the 3×Tg-AD mouse model was regionally variable and followed a mediolateral axis of involvement that was greatest in the fastigial nucleus, lesser in the interpositus and negligible in the dentate nucleus. A sole and low intraperitoneal dose of scFv-h3D6 protected 3×Tg-AD DCN neurons from death [cf. mAbs. 2013; 5(5):660-664].

The elongation mutants described herein also show effects in the same model (cf. Giménez-Llort L, Rivera-Hernandez G., Marin-Argany M., Sánchez-Quesada, J L & Villegas, S. Early intervention in the 3×Tg-AD mice with an amyloid β-antibody fragment ameliorates first hallmarks of Alzheimer disease mAbs 5:5, 665-677 2013

### E) In Vivo Experiments

1. Materials and methods

New Zealand White Rabbits were immunized with scFv-h3D6 or scFv-h3D6 elongation mutants to obtain a polyclonalantibody, as extensively described in the literature. This antibody was used to evaluate

the term in which a 100-μg single dose, administered intraperitoneally as reported effective in the literature, is detectable in 3×Tg-AD females.

Two mouse strains from The Jackson Laboratory were used:

B6 129SF2J mice (Genetic background)

3×Tg-AD (MMRRC034830 B6) mice (AD model). Transgenes: human APPswe, tauP301L and PS1M146V

2. ScFv-h3D6 or scFv-h3D6 Elongation Mutants Intraperitoneal Treatments:
2a. Pharmacokinetics in Blood and Brain:
a. Two groups of four 5-month-old 3×Tg-AD females were treated with saline or a single 100-μg dose of scFv-h3D6 or scFv-h3D6 elongation mutants, and serum was analyzed at different time periods by ELISA.
b. Six groups of four 5-month-old 3×Tg-AD females were treated with saline or a single 100-μg dose of scFv-h3D6 or scFv-h3D6 elongation mutants. Then, two groups (treated and untreated) were slaughtered at the time of its highest presence in serum, another two groups once undetectable in serum, and the remaining two after some days. Several areas were dissected (cortex, hippocampus, olfactory bulb, amygdale and cerebellum) to obtain protein extracts that were analyzed by ELISA for scFvh3D6 quantification.
2b. Determining the Therapeutic Window:

Once the treatment schedule was established, ten groups of four 5-month-old 3×Tg-AD females were treated with a different dose of scFv-h3D6 or scFv-h3D6 elongation mutants (0, 50, 100, 200, 500 μg), together with ten control groups (genetic background), and were monitored by MRI/MRS after each injection up to 18 months. At the end-point (18 months) animals were slaughtered to:

(i) Dissect several areas (cortex, hippocampus, olfactory bulb, amygdale and cerebellum) to perform protein extracts; determine the presence of Aβ-oligomers and LRP-1 by Western-blot (WB); and quantify the concentration of scFv-h3D6, apoE, and apoJ by ELISA, in order to determine the clearance mechanism.
(ii) Obtain a set of matched brain sections from the cortex, hippocampus, olfactory bulb, amygdale and cerebellum (10-μm slides) for (a) histochemical studies (IC) [to determine clearance (thioflavin S for amyloid plaques); examine the immune system's activation (lectine from Lycopersicon esculentum for microglia); and examine tissular architecture (Nissl for perikaryons)]; and (b) Immunohistochemical studies (IHC) with several antibodies [to determine clearance mechanism (anti-scFv-h3D6, -Aβ-oligomers, -apoE, -apoJ, -LRP-1 and -PSD-95); examine inflammation (anti-GFAP for astrogliosis (neuron injury and inflammation signaling); for cell-counting at different areas [anti-neurofilament H (pyramidal neurons in the cortex, large neurons in the Deep Cerebellar Nuclei, and Purkinje cells in the cerebellum cortex), -calretinin (bipolar cells and double-bouquet cells in the cortex, and small neurons in the Deep Cerebellar Nuclei), -parvalbumin (basket and Chandelier cells in the cortex), -somatostatin (Martinotti cells in the cortex), -NeuN (neuronal marker)].
(iii) Analyse serum for hepatotoxicity (AST, ALT), kidney function (urea, creatinine), inflammation (IL6).
(iv) Dissection of different organs: Liver, spleen, kidney and heart were weighed and prepared for IC with hematoxylin-eosin and Masson trichrome for tissular morphology characterization.
2c. Gender Comparison Experiments:

Eight groups of four 3×Tg-AD mice, four of them females and four males, all untreated, were used for matching amyloid burden. Comparison of 5, 8, 9 and 12 month-old animals was performed by obtaining Aβ extracts from different regions of the brain and quantification by ELISA. Females are known to develop the disease earlier and stronger than the males. Four groups of four 3×Tg-AD mice (5-month-old 3×Tg-AD females, treated and untreated, and matched 3×Tg-AD males, treated and untreated) were treated at the effective dose, longitudinally following its effect at two time-periods, and slaughtered as indicated for the determination of the therapeutic window.

### 3. In Vivo Nuclear Magnetic Resonance:

A Biospec (7T) allowing both imaging and proton spectroscopy was used to obtain a set of measurements, provided at months 5, 7, 9, 12, 15, and 18, for the determination of the therapeutic window, and at months 5 and 7 for females and 9 and 11 for males for gender comparison, in order to assess the evolution of neurodegeneration by diffusion tensor imaging and quantitative T2 mapping, as well as to image the evolution of amyloid plaques by MRI based on T2 and T2*, and quantitative T2 mapping. In addition the Biospec was used to rule out any possible occurrence of vasogenic edema by MRI diffusion coefficient measurements. Magnetic resonance spectroscopy was additionally used to quantify myo-inositol levels (as an indicator of astrogliosis), N-acetyl aspartate (as an indicator of the number of neurons) and glutamate (as an indicator of cell death).

Apart from the importance of improving molecules with therapeutic potential for AD treatment, these results from the scFv elongation mutants C1, C2 and C3 of the present invention can be extrapolated to other scFv molecules of therapeutic interest.

1. An isolated single chain variable fragment (scFv) comprising variable regions of the heavy (V_{H}) and light chains (V_{L}) of a monoclonal antibody, particularly a human or humanized monoclonal antibody, characterized in that the C-terminal end of said light chain is elongated by:
i) an amino acid residue, or
ii) a polypeptide having at least 2 amino acid residues.

2. An isolated single chain variable fragment according to claim 1 comprising variable regions of the heavy (V_{H}) and light chains (V_{L}) of humanized monoclonal antibody mAb-h3D6, characterized in that the C-terminal end of said light chain is elongated by:
i) an amino acid residue, or
ii) a polypeptide having at least 2 amino acid residues

3. An isolated single chain variable fragment according to claim 2 wherein the single chain variable fragment aggregates with the Aβ₁₋₄₂ peptide to form worm-like fibrils.

4. The single chain variable fragment according to claim 1, wherein the C-terminal end corresponds to lysine residue 107 of the light chain (V_{L}-K107) of the wild-type mAb-h3D6 using the Kabat Numbering Scheme.

5. The single chain variable fragment according to claim 1, wherein the C-terminal end of said single chain variable fragment is elongated by a glycine residue, a lysine residue, a threonine residue, a serine residue or an arginine residue.

6. The single chain variable fragment according to claim 1, wherein the C-terminal end of said single chain variable fragment is elongated by an arginine-threonine dipeptide.

7. The single chain variable fragment according to claim 5 represented by SEQ.ID.NO: 2 or SEQ. ID. NO: 3.

8. The single chain variable fragment according to claim 6 represented by SEQ.ID.NO: 4.

9. A nucleotide sequence encoding the single chain variable fragment according to claim 1.

10. A vector comprising the nucleotide sequence according to claim 9.

11. A host cell expressing the nucleotide sequence according to claim 9.

12. An isolated single chain variable fragment according to claim 1 for use as medicament.

13. An isolated single chain variable fragment according to claim 1 for use in the prevention and/or treatment of Alzheimer's disease in a patient.

14. Use of an isolated single chain variable fragment according to claim 1 in the manufacture of a medicament for the prevention and/or treatment of Alzheimer's disease.

15. A pharmaceutical composition comprising at least an isolated single chain variable fragment according to claim 1, and, optionally, at least a second active ingredient and/or at least an inert ingredient such an excipient and/or carrier.

16. A method of prevention or treatment of a patient in risk of suffering, or already diagnosed as suffering, Alzheimer disease that comprises the administration to said patient of an effective amount of the scFv elongation mutants of claim 1.

17. A method of prevention or treatment of a patient in risk of suffering, or already diagnosed as suffering, Alzheimer disease that comprises the administration to said patient of an effective amount of the scFv elongation mutants of the pharmaceutical composition of claim 15 comprising the same.


SEQUENCE LISTING (11 sequences)

SEQ ID NO: 1 - protein, 246 aa, Artificial Sequence
  derived from mAb-h3D6.v2
EVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSSGGGGSGGGGSGGGGSDVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIK

SEQ ID NO: 2 - protein, 247 aa, Artificial Sequence
  Light chain glycine108 elongation mutant (C1)
EVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSSGGGGSGGGGSGGGGSDVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKG

SEQ ID NO: 3 - protein, 247 aa, Artificial Sequence
  Light chain arginine108 elongation mutant (C2)
EVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSSGGGGSGGGGSGGGGSDVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKR

SEQ ID NO: 4 - protein, 248 aa, Artificial Sequence
  Light chain arginine108threonine109 elongation mutant (C3)
EVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSSGGGGSGGGGSGGGGSDVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKRT

SEQ ID NO: 5 - DNA, 738 bp, Artificial Sequence
  Wild-Type scFv-3D6h without cloning artefact nor stop codon
gaagtgcagctgctggaaagcggcggcggcctggtgcagccgggcggcagcctgcgcctgagctgcgcggcgagcggctttacctttagcaactatggcatgagctgggtgcgccaggcgccgggcaaaggcctggaatgggtggcgagcattcgcagcggcggcggccgcacctattatagcgataacgtgaaaggccgctttaccattagccgcgataacagcaaaaacaccctgtatctgcagatgaacagcctgcgcgcggaagataccgcggtgtattattgcgtgcgctatgatcattatagcggcagcagcgattattggggccagggcaccctggtgaccgtgagcagcggcggcggcggcagcggcggcggcggcagcggcggcggcggcagcgatgtggtgatgacccagagcccgctgagcctgccggtgaccccgggcgaaccggcgagcattagctgcaaaagcagccagagcctgctggatagcgatggcaaaacctatctgaactggctgctgcagaaaccgggccagagcccgcagcgcctgatttatctggtgagcaaactggatagcggcgtgccggatcgctttagcggcagcggcagcggcaccgattttaccctgaaaattagccgcgtggaagcggaagatgtgggcgtgtattattgctggcagggcacccattttccgcgcacctttggccagggcaccaaagtggaaattaaa

SEQ ID NO: 6 - DNA, 741 bp, Artificial Sequence
  Light chain glycine108 elongation mutant (C1)
gaagtgcagctgctggaaagcggcggcggcctggtgcagccgggcggcagcctgcgcctgagctgcgcggcgagcggctttacctttagcaactatggcatgagctgggtgcgccaggcgccgggcaaaggcctggaatgggtggcgagcattcgcagcggcggcggccgcacctattatagcgataacgtgaaaggccgctttaccattagccgcgataacagcaaaaacaccctgtatctgcagatgaacagcctgcgcgcggaagataccgcggtgtattattgcgtgcgctatgatcattatagcggcagcagcgattattggggccagggcaccctggtgaccgtgagcagcggcggcggcggcagcggcggcggcggcagcggcggcggcggcagcgatgtggtgatgacccagagcccgctgagcctgccggtgaccccgggcgaaccggcgagcattagctgcaaaagcagccagagcctgctggatagcgatggcaaaacctatctgaactggctgctgcagaaaccgggccagagcccgcagcgcctgatttatctggtgagcaaactggatagcggcgtgccggatcgctttagcggcagcggcagcggcaccgattttaccctgaaaattagccgcgtggaagcggaagatgtgggcgtgtattattgctggcagggcacccattttccgcgcacctttggccagggcaccaaagtggaaattaaaggt

SEQ ID NO: 7 - DNA, 741 bp, Artificial Sequence
  Light chain arginine108 elongation mutant (C2)
gaagtgcagctgctggaaagcggcggcggcctggtgcagccgggcggcagcctgcgcctgagctgcgcggcgagcggctttacctttagcaactatggcatgagctgggtgcgccaggcgccgggcaaaggcctggaatgggtggcgagcattcgcagcggcggcggccgcacctattatagcgataacgtgaaaggccgctttaccattagccgcgataacagcaaaaacaccctgtatctgcagatgaacagcctgcgcgcggaagataccgcggtgtattattgcgtgcgctatgatcattatagcggcagcagcgattattggggccagggcaccctggtgaccgtgagcagcggcggcggcggcagcggcggcggcggcagcggcggcggcggcagcgatgtggtgatgacccagagcccgctgagcctgccggtgaccccgggcgaaccggcgagcattagctgcaaaagcagccagagcctgctggatagcgatggcaaaacctatctgaactggctgctgcagaaaccgggccagagcccgcagcgcctgatttatctggtgagcaaactggatagcggcgtgccggatcgctttagcggcagcggcagcggcaccgattttaccctgaaaattagccgcgtggaagcggaagatgtgggcgtgtattattgctggcagggcacccattttccgcgcacctttggccagggcaccaaagtggaaattaaacgt

SEQ ID NO: 8 - DNA, 744 bp, Artificial Sequence
  Light chain arginine108threonine109 elongation mutant (C3)
gaagtgcagctgctggaaagcggcggcggcctggtgcagccgggcggcagcctgcgcctgagctgcgcggcgagcggctttacctttagcaactatggcatgagctgggtgcgccaggcgccgggcaaaggcctggaatgggtggcgagcattcgcagcggcggcggccgcacctattatagcgataacgtgaaaggccgctttaccattagccgcgataacagcaaaaacaccctgtatctgcagatgaacagcctgcgcgcggaagataccgcggtgtattattgcgtgcgctatgatcattatagcggcagcagcgattattggggccagggcaccctggtgaccgtgagcagcggcggcggcggcagcggcggcggcggcagcggcggcggcggcagcgatgtggtgatgacccagagcccgctgagcctgccggtgaccccgggcgaaccggcgagcattagctgcaaaagcagccagagcctgctggatagcgatggcaaaacctatctgaactggctgctgcagaaaccgggccagagcccgcagcgcctgatttatctggtgagcaaactggatagcggcgtgccggatcgctttagcggcagcggcagcggcaccgattttaccctgaaaattagccgcgtggaagcggaagatgtgggcgtgtattattgctggcagggcacccattttccgcgcacctttggccagggcaccaaagtggaaattaaacgtacc

SEQ ID NO: 9 - protein, 249 aa, Artificial Sequence
  Wild-Type scFV-3D6h with GAM cloning artefact
GAMEVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSSGGGGSGGGGSGGGGSDVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIK

SEQ ID NO: 10 - DNA, 753 bp, Artificial Sequence
  Wild-Type scFV-3D6h with GAM cloning artefact and stop codon
cgcgccatggaagtgcagctgctggaaagcggcggtggtctggtgcagccgggcggtagcctgcgtctgagctgcgccgcgagcggctttacctttagcaactatggcatgagctgggtgcgtcaggcgccgggcaaaggcctggaatgggttgcgagcattcgtagcggcggcggccgtacctattatagcgataacgtgaaaggtcgctttaccattagccgtgataatagcaaaaacaccctgtatctgcagatgaatagcctgcgtgccgaagataccgcggtgtattattgcgtgcgttatgatcattatagcggtagcagcgattattggggtcagggtaccctggttaccgtgagcagcggcggcggtggcagcggcggcggtggtagcggtggcggtggtagcgatgtggtgatgacccagagcccgctgagcctgccggtgaccccgggtgaaccggcgagcattagctgcaaaagcagccagagcctgctggatagcgatggcaaaacctatctgaattggctgctgcagaaaccgggccagagcccgcagcgcctgatctatctggtgagcaaactggatagcggcgtgccggatcgcttcagcggtagcggcagcggtaccgattttaccctgaaaattagccgtgtggaagcggaagatgttggtgtgtattattgctggcagggtacccattttccgcgtacctttggccagggcaccaaagtggaaatcaaataataa

SEQ ID NO: 11 - DNA, 6401 bp, Artificial Sequence
  pETM13_HIS_TRX_GS_TEV_GFP
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccggtaccttacttgtacagctcgtccatgccgagagtgatcccggcggcggtcacgaactccagcaggaccatgtgatcgcgcttctcgttggggtctttgctcaggccggactggtagctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcgggcgaagcactgcaggccgtagccgaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcactttgcccatggcgccctgaaaataaagattctcagaaccactgccagatccggccaggttagcgtcgaggaactctttcaactgacctttagacagtgcacccactttggttgccgccacttcaccgtttttgaacagcagcagagtcgggataccacggatgccatatttcggcgcagtgccagggttttgatcgatgttcagttttgcaacggtcagtttgccctgatattcgtcagcgatttcatccagaatcggggcgatcattttgcacggaccgcaccactctgcccagaaatcgacgaggatcgccccgtccgctttgagtacatccgtgtcaaaactgtcgtcagtcaggtgaataattttatcgctcatggggtgatggtgatggtgatgtttcatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca